SEQUENCE LISTING (3 sequences)

SEQ ID NO: 1 - protein, 11 aa, Artificial Sequence
  synthetic polypeptide
GDSVKKTKPRH

SEQ ID NO: 2 - protein, 15 aa, Artificial Sequence
  synthetic polypeptide
HQAVRKTAEKPKKKL

SEQ ID NO: 3 - protein, 15 aa, Artificial sequence
  synthetic polypeptide
LTNFVTNGKVERYTA

(19) United States
(12) Patent Application Publication
(10) Pub. No.: US 2012/0115132 A1
(43) Pub. Date: May 10, 2012

(54) IDENTIFICATION OF CENTROMERE SEQUENCES USING CENTROMERE ASSOCIATED PROTEINS AND USES THEREOF

(75) Inventors: Gregory P. COPENHAVER, Chapel Hill, NC (US); Helge Zieler, Del Mar, CA (US); Daphne Preuss, Chicago, IL (US)

(73) Assignee: CHROMATIN, INC., Chicago, IL (US)

(21) Appl. No.: 12/940,931

(22) Filed: Nov. 5, 2010

(51) Int. Cl. C12Q 1/68; C07H 21/00; C07H 21/04; C12N 1/00

(52) U.S. Cl. 435/6.11; 435/6.1; 435/243; 536/23.1; 536/23.2; 536/24.5; 435/6.19

(57) ABSTRACT

The present invention is directed to methods of centromere discovery using centromere-associated proteins in a variety of experimental formats. The methods of the invention can be used on any organism, and include using Cal1, Cbf1, Cbf3, Cbf5, CenH3 (Cenp-A), Cenp-B, Cenp-C, Cenp-D, Cenp-E, Cenp-F, Cenp-G, Cenp-H, Cenp-I, Cenp-K, Cenp-L, Cenp-M, Cenp-N, Cenp-O, Cenp-P, Cenp-Q, Cenp-R, Cenp-S, Cenp-T, Cenp-U, Cenp-V, Cenp-W, Chd1, Chp1, cohesin, condensin, Dnmt3b, Fact, Gcn5p, H2A.Z, Haspin, Hjurp, HP1, Hst4, Ima1, Incep, Ino80, Kms2, Knl-2, Mif2, Mis6, Np95, Pich, Sad1, Scm3, Shugoshin, Sim3, Skp1, Sororin, Survivin, Tas3, ZW10, and homologs thereof to identify centromere sequences. The invention is also directed to artificial chromosomes comprising centromeres made according to the methods of the invention, as well as to cells comprising such artificial chromosomes.

### CROSS REFERENCE TO RELATED APPLICATION

NONE

### FIELD OF THE INVENTION

The present invention relates to methods for identifying centromeric sequences that are useful, for example, in constructing artificial chromosomes comprising centromeres comprising such identified centromeric sequences, and cells and organisms comprising such artificial chromosomes. The present invention also discloses centromeric sequences useful, for example, in constructing artificial chromosomes for use in algae.

### GOVERNMENT SUPPORT

Not applicable.

### COMPACT DISC FOR SEQUENCE LISTINGS AND TABLES

Not applicable.

### BACKGROUND OF THE INVENTION

Agricultural and aquacultural crops have the potential to meet escalating global demands for affordable and sustainable production of food, fuels, fibers, therapeutics, and biomaterials (Herrera, 2004). While integrative plant and algal transformation techniques can often meet these needs by safely introducing novel genes into plant chromosomes, they have limited efficiency and can disrupt the host genome (note—algae are a phylogenetically diverse group of organisms that include members in two kingdoms (Plantae and Protista), for simplicity algae is included under the term “plant” in this application). Typically, biological delivery of DNA carried on an Agrobacterium Ti plasmid (T-DNA), or biolistic delivery of small DNA-coated particles is used to transfer and integrate desired genes into a host plant chromosome (Lorence and Verpoorte 2004). Integration at random sites can result in unpredictable transgene expression due to position effect variegation, variable copy number from multiple (including tandem) integrations, and frequent loss of gene integrity as a result of intragenic transgene insertion (Birch, 1997; Lorence and Verpoorte, 2004). Transgene integration also results in genetic linkage of the introduced genes to portions of the genome that encode loci that can confer undesired phenotypes (a phenomenon known as linkage drag), adding complexity when the transgenic locus is used for downstream breeding purposes (Walker et al., 2002; Yin et al., 2004). In addition, integrative technologies have typically been limited in the length of DNA that they can efficiently deliver. Recent advances in gene integration technologies have aimed to surmount some of these difficulties. For example, zinc finger-mediated homologous recombination or site-specific recombination could eliminate the unpredictable expression that results from random insertion into the plant genome, but still suffer from the linkage drag problem (Gilbertson, 2003; Kumar et al., 2006). In addition, combining binary T-DNA elements with bacterial artificial chromosome (BAC) technology to produce BiBACs has the potential to introduce larger DNA fragments into the host genome (Hamilton et al., 1996; He et al., 2003). In contrast to these systems, minichromosomes (MCs) remain separate (autonomous) from the host chromosomes and have the capacity to carry large transgenic payloads. Thus they provide an alternative approach with important benefits including: predictability of expression, no linkage drag, no disruption of the host chromosomes and increased flexibility in the size of the transgene cassette. Indeed, although precise integration into host chromosomes has long been a routine technique in Saccharomyces cerevisiae, the facile properties of autonomous vectors often make them a preferred choice for numerous applications, including commercial-scale protein production.

The first eukaryotic MCs used a simple centromere (CEN) sequence from the budding yeast S. cerevisiae, incorporated into versatile circular and linear yeast artificial chromosome (YAC) vectors (Burke et al., 1987; Clarke and Carbon, 1980). These yeast vectors were used to define a 125-bp DNA fragment sufficient for mitotic and meiotic centromere function (Cottarel, Shero et al. 1989). While circular CEN vectors are most useful for carrying smaller DNA fragments, YAC vectors can carry megabase quantities of DNA and are convenient for manipulating large fragments of DNA (Larin et al., 1991). Similarly, with carrying capacities of hundreds of kb, human artificial chromosomes (HACs) provide advantages over other in vitro-assembled vectors used in human cell transfection (Kuroiwa et al., 2000). HACs containing tandem repeats of a centromeric 171-bp alpha satellite sequence can be maintained either as circular or linear, telomere-containing, episomes (Ebersole et al., 2000; Harrington et al., 1997; Ikeno et al., 1998; Schueler et al., 2001; Tsuduki et al., 2006).

DNA sequences that can form stable MCs are able to recapitulate centromere functions de novo by recruiting essential DNA binding proteins and epigenetic modifications. In human cells, different repetitive DNA (satellite) arrays vary in their ability to efficiently form HACs, based on their monomer sequence, chromosomal origin, array length, higher-order structure, and even vector composition (Grimes et al., 2002; Mejia et al., 2002; Ohzeki et al., 2002; Okamoto et al., 2007). These DNA sequences recruit centromere binding protein A (CENP-A), which substitutes for histone H3 to form centromeric nucleosomes. CENP-A orthologs are known to mark active centromeres in a phylogenetically diverse set of organisms including S. cerevisiae (Cse4p), Schizosaccharomyces pombe (Cnp1), Drosophila melanogaster (Cid), Arabidopsis thaliana (HTR12), Zea mays (CENH3), and Homo sapiens (CENP-A) (Malik and Henikoff, 2001; Meluh et al., 1998; Palmer et al., 1987; Takahashi et al., 2000; Talbert et al., 2002; Zhong et al., 2002). CENP-A complexes are maintained through mitosis and meiosis (Schatten et al., 1988), resulting in an epigenetic mark that is important in perpetuating centromere activity. Evidence for this role in centromere maintenance comes from human neocentromeres (Lo et al., 2001), where, at a very low frequency, aberrant ectopic centromeres are nucleated in regions that lack satellite DNA. Once formed, these neocentromeres are efficiently maintained.

The ability to form centromeres on naked DNA depends on cell type in mammalian systems. Indeed, HAC formation has been most commonly demonstrated in HT1080 fibrosarcoma cells. Yet once established, HACs can be transferred to other mammalian cell types, where they are stably maintained (Suzuki et al., 2006).

Maize centromeres are structurally similar to mammalian centromeres in that they contain repetitive sequences though there is no sequence similarity between the repeats in the different species. For example, analogous to the tandem arrays of 171-bp alpha satellite found in human centromeres, large tandem arrays of the 156-bp maize CentC satellite bind to CENP-A (Ananiev et al., 1998; Nagaki et al., 2003; Zhong et al., 2002). In maize, these satellite arrays are often interrupted by CRM, a centromere-specific retroelement that also binds CENP-A (Zhong et al., 2002). Some maize varieties also have supernumerary B chromosomes with a distinct centromere satellite sequence, ZmBs (Alfenito and Birchler, 1993; Jin et al., 2005). These B chromosomes lack essential genes, and thus have been particularly useful for discerning the relationship between centromere structure and meiotic transmission (Kaszas et al., 2002; Kato et al., 2005; Phelps-Durr and Birchler, 2004). A series of deletion derivatives of natural B chromosomes, derived from an A-B translocation event, showed a strong dependence on centromere size—the smallest functional derivative contained a 110-kb centromere and resulted in a meiotic transmission rate of 5%, yet showed a high stability in mitosis (Phelps-Durr and Birchler 2004). More recently, telomere-mediated chromosomal truncation was used to generate deletion derivatives from both A and B maize chromosomes [40]. Transgenes carried on these derivative chromosomes (or “engineered MCs”) were expressed and meiotic inheritance ranged from 12% to 39% (Yu et al., 2007). While this telomere-truncation approach can deliver both transgenes and sequences that promote site-directed integration, its utility for commercial applications can be limited—most commercial maize hybrids lack B chromosomes.

Carlson et al. (2007) have described autonomous MCs that do not rely on alteration of endogenous chromosomes (Carlson, Rudgers et al. 2007). Carlson et al. constructed plasmids carrying maize centromeric repeats, delivered purified constructs to embryogenic maize tissue, and assessed their ability to promote the formation of maize minichromosomes (MMC5). MMC1 was characterized in detail; this CentC-based construct contained 19 kb of centromeric DNA and conferred efficient mitotic and meiotic inheritance through at least four generations when introduced into plant cells.

Making artificial chromosomes often requires centromeric sequences specific to a target organism, as sequences from a related organism sometimes do not work efficiently in establishing centromere function (Kitada et al., 1997; Pribylova et al., 2007) Identification of centromeres has been pursued in several organisms by searching for repetitive DNA or methylated DNA followed by labeling studies to determine whether the identified sequences hybridize to the centromere region of chromosomes, and/or functional studies to determine whether the identified sequence(s) function as centromeres (see, for example, U.S. Pat. No. 7,456,013, WO 08/112,972).

Other work has attempted to use centromere-associated proteins to map centromeres and attempted to determine the involvement of particular sequences in centromere function (Vafa and Sullivan 1997; Lo, Magliano et al. 2001; Zhong, Marshall et al. 2002; Alonso, Mahmood et al. 2003; Nagaki, Song et al. 2003; Nagaki, Talbert et al. 2003; Jin, Melo et al. 2004; Jin, Lamb et al. 2005; Nagaki and Murata 2005). For example, Jin Lamb et al. (2005) examined the centromere of the maize B chromosome, which contains several megabases of a B-specific repeat (ZmBs), a 156-bp satellite repeat (CentC), and centromere-specific retrotransposons (CRM elements). They observed that a small fraction of the ZmBs repeats interacts with CENH3, the histone H3 variant specific to centromeres. CentC, which marks the CENH3-associated chromatin in maize A-chromosome centromeres, is restricted to an approximately 700-kb domain within the larger context of the ZmBs repeats. Other analysis showed that the functional boundaries of the B centromere mapped to a relatively small CentC- and CRM-rich region that is embedded within multimegabase arrays of the ZmBs repeat, noting that the amount of CENH3 at the B centromere can be varied, but with decreasing amounts, the function of the centromere becomes impaired. Zhong, Marshall, et al. (2002) used antibodies against CENH3 to determine what centromeric DNA sequences are part of a functional centromere/kinetochore complex. CENH3 is a highly conserved protein that replaces histone H3 in centromeres and is thought to recruit many of the proteins required for chromosome movement. Zhong, Marshall et al. found that chromatin immunoprecipitation with anti-CENH3 antibodies co-precipitated CentC and CRM sequences. These references, however, did not use centromere-associated proteins for the isolation of large fragments of centromere DNA, or for the establishment of centromeres in artificial chromosomes.

Approaches to Identify Centromeric Sequences

A variety of molecular biology approaches have been used to isolate centromeric sequences from plants. These include (i) isolation of random, tandemly repeated genomic sequences by restriction digestion of genomic DNA, (ii) cloning of Cot DNA, (iii) isolation and cloning of hypermethylated DNA and (iv) discovery of repetitive sequences in genomic sequences present in Genbank and other public sequence repositories. In some organisms (Brassica sp., tomato), scientists have had great success in identifying the major centromeric sequences (Carlson, Rudgers et al. 2007) and U.S. Pat. Nos. 7,456,013, 7,227,057, 7235,716 and 7,226,782; in other species, however, such methods have been less immediately successful. Conserved centromere features other than sequence can be exploited to isolate centromere sequences from novel species. For example, CenH3 (known as CENP-A in humans) is a variant of the nucleosome protein histone H3 that is preferentially associated with centromeric chromatin. This protein differs from histone H3 in having longer and divergent N-terminal sequences. Antibodies raised against the unique N-terminal sequences of CenH3 have been used in some strategies for isolating centromere sequences from some species, for example, using chromatin immunoprecipitation (ChIP), followed by methods to detect the immoprecipitated DNA such as amplification of specific target sequences by PCR (ChIP-PCR) DNA sequencing (ChIP-seq) or application to a microarray (ChIP-chip). Because immunoprecipitation of chromatin typically results in isolation of non-specific sequences as well as the sequence(s) of interest, when used for centromere identification, it has been performed in conjunction with hybridization to chromosome spreads using fluorescence in situ hybridization (FISH) or comparisons with sequence motifs previously known to be associated or suspected of being associated with centromeres in the organism of interest (Nagaki, Talbert et al. 2003; Lee, Zhang et al. 2005) thus relying on prior knowledge of centromere-associated sequences.

Algae

Algae are a diverse group of photosynthetic organisms that are important in marine, freshwater, and some terrestrial ecosystems. The major groups of algae are the Chlorophyta (green algae), Rhodophyta (red algae), Glaucocystophyta, Euglenophyta, Chlorarachniophyta, Heterokontophyta, Haptophyta, Cryptophyta and the dinoflagellates (Bhattacharya and Medlin 1998). Older phylogenetic groupings included the prokaryotic cyanobacteria as algae but these are now considered bacteria. Algae have gained in importance commercially not only as a source of feed and chemicals, but also as a means to produce biofuels.

Green algae appear evolutionarily most closely related to plants, having the same pigments, chlorophyll a and b and carotenoids, cell wall macromolecules (e.g., cellulose), and storage product, starch.

Centromere identification in algae has been challenging. Unlike most plants described to date, some algal centromeres may be non-repetitive centromeres reminiscent of fungal centromeres, like those of the yeast Saccharomyces cerevisiae. For example, after observing that CENH3-containing nucleosomes constituted the kinetochore closely interacting with the nuclear envelope in the red algae Cyanidioschyzon merole, a 100% no-gap telomere-to-telomere sequencing effort was undertaken and analyzed. Instead of finding repeat structures reminiscent of higher plant centromeres, a single A+T-rich region was identified on each fully-sequenced chromosome, implying that the C. merole centromeres may be an A+T % “point” centromere, or alternatively, be comprised of non-repetitive heterogeneous DNA sequences (Maruyama, Matsuzaki et al. 2008). In 2006, the complete genome (20 chromosomes) for the unicellular green alga Ostreococcus tauri was sequenced and analyzed; the researchers noted very few repeat sequences suggesting that O. tauri may also have small non-repetitive centromeres. Adding to the suggested variety of centromere structures in algae, analysis of a contig in the green algae Chlorella vulgaris suggested the centromeres may be associated with bent DNA and retro-elements. Based on such contigs, Noutoshi et al also suggested designing a plant artificial chromosome based on C. vulgaris (Noutoshi, Arai et al. 1997).

Centromere binding proteins have been identified in algae. For example, CENH3 in Cyanidioschyzon merole (Maruyama, Kuroiwa et al. 2007); ZW10 in Phaeodactylum tricornutum (De Martino, Amato et al. 2009); and ZW10 in Thalassiosira pseudonana (De Martino, Amato et al. 2009). Several other centromere binding or centromere associated proteins are known in other organisms and it is anticipated that orthologous proteins exist in algae. Table 1 lists several such proteins.

TABLE 1
Examples of centromere binding/centromere associated proteins

| Protein | Reference |
|---|---|
| Cal1 | (Schittenhelm, Althoff et al. 2010) |
| Cbf1 | (Cai and Davis 1990) |
| Cbf3 | (Lechner and Carbon 1991) |
| Cbf5 | (Jiang, Middleton et al. 1993) |
| CenH3 (Cenp-A) | (Earnshaw and Migeon 1985) |
| Cenp-B | (Earnshaw and Migeon 1985) |
| Cenp-C | (Earnshaw and Migeon 1985) |
| Cenp-D | (Yen, Compton et al. 1991) |
| Cenp-E | (Yen, Compton et al. 1991) |
| Cenp-F | (Rattner, Rao et al. 1993) |
| Cenp-G | (He, Zeng et al. 1998) |
| Cenp-H | (Sugata, Munekata et al. 1999) |
| Cenp-I | (Nishihashi, Haraguchi et al. 2002) |
| Cenp-K | (Foltz, Jansen et al. 2006) |
| Cenp-L | (Foltz, Jansen et al. 2006) |
| Cenp-M | (Foltz, Jansen et al. 2006) |
| Cenp-N | (Foltz, Jansen et al. 2006) |
| Cenp-O | (Foltz, Jansen et al. 2006) |
| Cenp-P | (Foltz, Jansen et al. 2006) |
| Cenp-Q | (Foltz, Jansen et al. 2006) |
| Cenp-R | (Foltz, Jansen et al. 2006) |
| Cenp-S | (Foltz, Jansen et al. 2006) |
| Cenp-T | (Foltz, Jansen et al. 2006) |
| Cenp-U | (Foltz, Jansen et al. 2006) |
| Cenp-V | (Tadeu, Ribeiro et al. 2008) |
| Cenp-W | (Hori, Amano et al. 2008) |
| Chd1 | (Okada, Okawa et al. 2009) |
| Chp1 | (Doe, Wang et al. 1998) |
| cohesin | (Klein, Mahr et al. 1999) |
| condensin | (Hagstrom, Holmes et al. 2002) |
| Dnmt3b | (Okano, Bell et al. 1999) |
| Fact | (Foltz, Jansen et al. 2006) |
| Gcn5p | (Vernarecci, Ornaghi et al. 2008) |
| H2A.Z | (Greaves, Rangasamy et al. 2007) |
| Haspin | (Dai, Sullivan et al. 2006) |
| Hjurp | (Foltz, Jansen et al. 2009) |
| HP1 | (Saunders, Chue et al. 1993) |
| Hst4 | (Freeman-Cook, Sherman et al. 1999) |
| Ima1 | (King, Drivas et al. 2008) |
| Incep | (Cooke, Heck et al. 1987) |
| Ino80 | (Ogiwara, Enomoto et al. 2007) |
| Kms2 | (King, Drivas et al. 2008) |
| Knl-2 | (Maddox, Hyndman et al. 2007) |
| Mif2 | (Meluh and Koshland 1995) |
| Mis6 | (Saitoh, Takahashi et al. 1997) |
| Np95 | (Papait, Pistore et al. 2007) |
| Pich | (Baumann, Korner et al. 2007) |
| Sad1 | (King, Drivas et al. 2008) |
| Scm3 | (Stoler, Rogers et al. 2007) |
| Shugoshin | (Kitajima, Kawashima et al. 2004) |
| Sim3 | (Dunleavy, Pidoux et al. 2007) |
| Skp1 | (Connelly and Hieter 1996) |
| Sororin | (Diaz-Martinez, Gimenez-Abian et al. 2007) |
| Survivin | (Uren, Wong et al. 2000) |
| Tas3 | (Verdel, Jia et al. 2004) |
| ZW10 | (Williams, Gatti et al. 1996) |

### BRIEF SUMMARY OF THE INVENTION

In a first aspect, the invention is directed to methods of identifying a centromere sequence, comprising: (a) immunoprecipitating protein-DNA complexes from fragmented chromatin derived from at least one cell using an antibody to a centromere-associated protein; (b) separately sequencing individual nucleic acid molecules of a population of nucleic acid molecules isolated from the protein-DNA complexes; (d) calculating the frequency of occurrence of each nucleic acid sequence in the population; and (e) identifying a nucleic acid molecule sequence which has an increased frequency of occurrence in the population as a centromere sequence;

In a second aspect, the invention is directed to methods of identifying a centromere sequence, comprising: (a) fusing a centromere-associated protein with a DNA adenine methyltransferase to create a fusion protein; (b) expressing the fusion protein in at least one cell of interest; (c) isolating methylated DNA from the cell of interest; (d) separately sequencing the isolated methylated DNA; and (e) identifying the DNA which has an increased frequency of occurrence as a centromere sequence.

In a third aspect, the invention is directed to methods of identifying a centromere sequence, comprising: (a) fusing a centromere-associated protein with a protein that tightly binds to a chloroalkane resin to create a fusion protein; (b) expressing the fusion protein in at least one cell of interest; (c) isolating chromatin from the cell of interest and cross-linking the isolated chromatin; (d) isolating fusion protein/DNA complexes by passing the isolated, cross-linked chromatin over a chrloroalkane resin and reversing the cross-linking of the resin to disrupt the protein/DNA complexes; and (e) separately sequencing the isolated DNA; and (f) identifying the DNA which has an increased frequency of occurrence as a centromere sequence.

In a fourth aspect, the invention is directed to methods of identifying a centromere sequence, comprising: (a) labeling and isolating DNA from at least one cell of interest; (b) incubating the labeled and isolated DNA with a centromere-associated protein, forming centromere-associated protein/DNA complexes; (c) electrophoresing the mixture from step (b) to separate the centromere-associated protein/DNA complexes from unbound labeled DNA; (d) isolating slower-migrating DNA representing centromere-associated protein/DNA complexes; (e) isolating the DNA from the centromere-associated protein/DNA complexes; (f) separately sequencing the isolated DNA; and (g) identifying the DNA which has an increased frequency of occurrence as a centromere sequence.

In a fifth aspect, the invention is directed to methods of identifying a centromere sequence, comprising: (a) immobilizing a centromere-associated protein onto a substrate; (b) incubating labeled DNA isolated from at least one cell of interest with the centromere-associated protein; (c) isolating bound DNA; (d) separately sequencing the isolated DNA; and (e) identifying the DNA which has an increased frequency of occurrence as a centromere sequence.

In a sixth aspect, the invention is directed to methods of the first five aspects, further comprising, prior to sequencing the nucleic acid or DNA, separately amplifying individual nucleic acid molecules of a population of nucleic acid molecules isolated from the protein-DNA complexes; and wherein at least one cell is at least one plant, fungal, algal, or protist cell, wherein at least one algal cell is of the Chlorophyceae, Pluerastrophyceae, Ulvophyceae, Micromonadophyceae, or Charophytes class, for example, wherein at least one algal cell is a cell of an alga of the Dunaliellale, Volvocale, Chloroccale, Oedogoniale, Sphaerolpleale, Chaetophorale, Microsporale, or Tetrasporale orders, such as an alga cell that is an Amphora, Ankistrodesmus, Asteromonas, Botryococcus, Chaetoceros, Chlamydomonas, Chlorococcum, Chlorella, Cricosphaera, Crypthecodinium, Cyclotella, Dunaliella, Emiliania, Euglena, Haematococcus, Halocafeteria, Isochrysis, Monoraphidium, Nannochloris, Nannochloropsis, Navicula, Neochloris, Nitzschia, Ochromonas, Oedogonium, Oocystis, Ostreococcus, Pavlova, Phaeodactylum, Pleurochrysis, Pleurococcus, Pyramimonas, Scenedesmus, Skeletonema, Stichococcus, Tetraselmis, Thalassiosira or Volvox species. Alternatively, the at least one cell can be a fungal cell, such as of a chytrid, blastocladiomycete, neocallimastigomycete, zgomycete, trichomycete, glomeromycote, ascomycete, or basidiomycete.

In a seventh aspect, the invention is directed to the methods of the first five aspects, wherein the centromere-associated protein is selected from the group consisting of centromere proteins, centromere protein-recruitment proteins, and kinetochore proteins. Such centromere-associated proteins can be Cal1, Cbf1, Cbf3, Cbf5, CenH3 (Cenp-A), Cenp-B, Cenp-C, Cenp-D, Cenp-E, Cenp-F, Cenp-G, Cenp-H, Cenp-I, Cenp-K, Cenp-L, Cenp-M, Cenp-N, Cenp-O, Cenp-P, Cenp-Q, Cenp-R, Cenp-S, Cenp-T, Cenp-U, Cenp-V, Cenp-W, Chd1, Chp1, cohesin, condensin, Dnmt3b, Fact, Gcn5p, H2A.Z, Haspin, Hjurp, HP1, Hst4, Ima1, Incep, Ino80, Kms2, Knl-2, Mif2, Mis6, Np95, Pich, Sad1, Scm3, Shugoshin, Sim3, Skp1, Sororin, Survivin, Tas3, or ZW10, and homologs thereof.

In an eighth aspect, the invention is directed to methods of evaluating the centromere sequences identified by the methods of the invention. Such assays include those that assay for stable heritability of an artificial chromosome comprising the centromere sequence; or detects the presence of a selectable or nonselectable marker on an artificial chromosome comprising the centromere sequence; or detects the presence of the centromere sequence or a nucleic acid sequence linked thereto on an artificial chromosome.

In a ninth aspect, the invention is directed to recombinant nucleic acid molecule comprising a centromere sequence identified by the methods of the present invention. Such centromere sequence may not be adjacent to one or more sequences positioned adjacent to the centromere sequence in the genome from which the centromere sequence is derived.

In a tenth aspect, the invention is directed to artificial chromosomes, such as minichromosomes, comprising a centromere sequence identified by the methods of the invention. Such artificial chromosomes can further comprise selectable or nonselectable markers, or at least one gene encoding a structural protein, a regulatory protein, an enzyme, a ribozyme, an antisense RNA, an shRNA, or an siRNA.

In an eleventh aspect, the invention is directed to cells comprising an artificial chromosome made according to the methods of the present invention.

In a twelfth aspect, the invention is directed to methods of identifying an algal centromere sequence, comprising: (a) immunoprecipitating protein-DNA complexes from fragmented chromatin derived from at least one algal cell using an antibody to a centromere-associated protein; and (b) sequencing nucleic acid molecules isolated from the protein-DNA complexes to identify an algal centromere sequence. The method does not necessarily require the addition of a cross-linking agent prior to immunprecipitating protein-DNA complexes from the fragmented chromatin, or does not require hybridizing a nucleic acid molecule isolated from the immunoprecipitated protein-DNA complexes to one or more known centromere sequences. The at least one algal cell is at least one green, yellow-green, brown, golden brown, or red algal cell; the algal cell can be of the Chlorophyceae class, from the Dunaliellale, Volvocale, Chloroccale, Oedogoniale, Sphaerolpleale, Chaetophorale, Microsporale, or Tetrasporale order; a cell of an Amphora, Ankistrodesmus, Aster vmonas, Botryococcus, Chaetoceros, Chlamydomonas, Chlorococcum, Chlorella, Cricosphaera, Crypthecodinium, Cyclotella, Dunaliella, Emiliania, Euglena, Haematococcus, Halocafeteria, Isochrysis, Monoraphidium, Nannochloris, Nannochloropsis, Navicula, Neochloris, Nitzschia, Ochromonas, Oedogonium, Oocystis, Ostreococcus, Pavlova, Phaeodactylum, Pleurochrysis, Pleurococcus, Pyramimonas, Scenedesmus, Skeletonema, Stichococcus, Tetraselmis, Thalassiosira or Volvox species.

In a thirteenth aspect, the method of the twelfth aspect uses a centromere-associated protein selected from the group consisting of centromere proteins, centromere protein-recruitment proteins, and kinetochore proteins. Such centromere associated proteins include Cal1, Cbf1, Cbf3, Cbf5, CenH3 (Cenp-A), Cenp-B, Cenp-C, Cenp-D, Cenp-E, Cenp-F, Cenp-G, Cenp-H, Cenp-I, Cenp-K, Cenp-L, Cenp-M, Cenp-N, Cenp-O, Cenp-P, Cenp-Q, Cenp-R, Cenp-S, Cenp-T, Cenp-U, Cenp-V, Cenp-W, Chd1, Chp1, cohesin, condensin, Dnmt3b, Fact, Gcn5p, H2A.Z, Haspin, Hjurp, HP1, Hst4, Ima1, Incep, Ino80, Kms2, Knl-2, Mif2, Mis6, Np95, Pich, Sad1, Scm3, Shugoshin, Sim3, Skp1, Sororin, Survivin, Tas3, or ZW10, and homologs thereof.

In a fourteenth aspect, the method of the twelfth aspect can further comprise amplifying the nucleic acid molecules isolated from the immunoprecipitated protein-DNA complexes prior to sequencing.

### BRIEF DESCRIPTION OF SEVERAL VIEWS OF THE DRAWING

Not applicable

### DETAILED DESCRIPTION OF THE INVENTION

### I. Introduction

The present invention solves the problem of identifying functional centromeric (CEN) sequences by exploiting the functional relationship between chromatin-binding molecules and CENs. These methods permit the direct identification of functional CEN sequences of various sizes by virtue of binding to the plant centromere-associated proteins (CAPs).

In some methods of the present invention, chromatin from a target organism is fragmented. This fragmented chromatin harbors CAP-CEN sequence complexes (“CAP complexes”). An antibody or other reagent that binds to a CAP in the complex is added, and CAP complexes precipitated. This purification allows for the isolation of bound DNA from the CAP complexes, providing specific DNA sequence that can be used to identify and describe functional CEN sequences. For example, individual nucleic acid molecules of a population of nucleic acid molecules isolated from the protein-DNA complexes can be sequenced, and the sequence analyzed for an enrichment of specific sequences, thus correlating to CEN sequences. Alternatively, the isolated DNA can be used as probes of libraries of genomic DNA to identify those segments of DNA that harbor CEN sequences. In any case, the identified candidate CEN sequences can be subjected to a battery of tests to confirm centromere function, such as the ability of the sequence to confer autonomy to an artificial chromosome construct. In one embodiment, antibodies or other molecules that specifically bind to CAP CenpA/CenH3 are used. In other embodiments, antibodies or other molecules that specifically bind to CAP CenpB are used. In other embodiments, antibodies or other molecules that bind to the CAPs listed in Table 1 are used.

In other embodiments, the CAP itself is used to screen DNA sequences for their ability to specifically be bound by the CAP. CAPs can be isolated from target cells, or produced using recombinant methods. The CAPs can then be used to screen isolated DNA, or genomic DNA, or libraries of DNA to identify putative CEN sequences. Techniques including EMSA and Southwestern blotting would be useful in this approach.

In other embodiments, the CAP is fused to a protein or peptide. The protein fusion is then incubated or otherwise exposed to isolated DNA, or genomic DNA, or libraries of DNA to identify putative CEN sequences. In this approach the peptide or protein fused to the CAP is used as a tag to isolate it the CAP/DNA complex. Techniques such as Halo-tagging (Promega Corporation; Madison, Wis.) or DamID are useful in this approach.

In human cells, the ability of alpha-satellite repeats to bind CenpB correlates with the de novo centromere function of these repeats. Due to the conserved nature of CenpB proteins, the same is expected to be true in plants and algae. In human cells and plants, association of centromere sequences with the CAP CenH3 correlates very closely with centromere function. The invention discloses methods that exploit the specific the association of CAPs with centromere sequences as a method to isolate sequences with centromere function, such as from plants, fungi and algae. In the methods of the invention, while exemplified with specific CAPs, any protein that specifically associates directly or indirectly with a chromosome's centromere or kinetochore, such as those listed in Table 1, can be used to either screen DNA directly, or to be used to make antibodies or other CAP-binding molecules for isolation of CAP/DNA complexes.

There are many ways that such a screen or purification could be done, including: interaction of CAP with random genomic sequences or with pooled, cloned, or otherwise selected DNA sequences in solution, followed by immunoprecipitation ChIP), and cloning of the precipitated sequences and their characterization by sequencing, or use of immunoprecipitated sequences as probes for blots or genomic libraries; by immobilization of selected DNA sequences (either purified or cloned, single or pooled) and use of the CAP as a protein probe to determine that DNA sequences bind CAP. It may also be desirable to perform the isolation of the CAP/DNA complex during specific parts of the cell cycle or during specific developmental stages or from specific tissues of sub-sets of cells. For example, cells undergoing cell division (mitotic or meiotic) or cells from reproductive tissue may be enriched for CAP/DNA interactions. Isolation or identification of the desired sequences, after binding CAP, can be accomplished by using CAP-specific antiserum (monoclonal or polyclonal), or by epitope tagging a CAP prior to expression and purification, and detection with an antibody or antiserum specific to the epitope tag. These methods result in the identification of sequences of any length, including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 171, 180 bp long. These methods may also result in the identification of sequences ranging from 100 to 150, 150 to 200, 200 to 250, 250 to 300, 300 to 350, 350 to 400, 400 to 450, 450 to 500, 500 to 600, 600 to 700, 700 to 800, 800 to 900, 900 to 1000, 1000 to 1500, 1500 to 2000, 2000 to 2500, 2500 to 3000, 3000 to 3500, 3500 to 4000, 4000 to 4500, 4500 to 5000, 5000 to 6000, 6000 to 7000, 7000 to 8000, 8000 to 9000, 9000 to 10,000, 10,000 to 15,000, 15,000 to 20,000, 20,000 to 25,000, 25,000 to 30,000, 30,00 to 40,000, 40,000 to 50,000 bp and sequences longer than 50,000 bp. or other types of genomic DNA cloned into vectors capable of carrying large-inserts, that bind CAP and therefore are likely to have de novo centromere function.

In other embodiments of the invention it may be multiple CAPs can be used to identify candidate centromere sequences. In this approach a first CAP (e.g. CenH3) is used to isolate a first pool of candidate centromere sequences as described above. Subsequently, or in parallel, a second CAP (e.g. Cenp-B) is used to isolate a second pool of candidate centromere sequences. Each pool of sequences is then compared, for example by sequence alignment, to determine if there is overlap between the two pools. Sequences that are represented in both pools may have a higher probability of functioning as centromeres by virtue of their association with multiple CAPs. This approach can be used with 2, 3, 4, 5, 6, 7, 8, 9, 10, 11, 12, 13, 14, 15, 16, 17, 18, 19, or more CAPs. In a related approach it proteins that are known to not bind centromere sequences (non-CAP) are useful as controls or to define background levels of non-specific binding.

In other embodiments of the invention CAPs decorated with posttranslational modifications are used to identify centromere sequences. Useful posttranslational modifications include but are not limited to: acetylation, formylation, lipolation, myristoylation, palmitoylation, methylation, isoprenylation, farnesylation, geranylgeranylation, amidation, arginylation, polyglutamylation, polyglycylation, gamma-carboxylation, glycosylation, glypiation, hydroxylation, iodination, adenylation, ADP-ribosylation, flavin attachment, nitrosylation, S-glutathionylation, oxidation, phosphopantetheinylation, phosphorylation, pyroglutamate formation, sulfation, selenoylation, and glycation.

### II. Definitions

Unless defined otherwise, all technical and scientific terms used herein have the same meaning as commonly understood by one of ordinary skill in the art to which this invention is related. The following terms are defined for purposes of the invention as described herein.

“About” or “approximately” when referring to any numerical value are intended to mean a value of plus or minus 10% of the stated value.

“Algae” means any kind of alga, including, for example those from the phyla Chlorophyta (green algae), Rhodophyta (red algae), Glaucocystophyta, Euglenophyta, Chlorarachniophyta, Heterokontophyta, Haptophyta, Cryptophyta and the dinoflagellates, microalgae, diatoms, cyanobacteria and macroalgae (e.g., seaweed), and those listed below. Other types of alga are known to those of skill in the art and can be used with the invention. The following are examples of algae: dinoflagellates, including, for example, Crypthecodinium cohnii; thraustochytrids, including, for example, Thraustochytrium spp., Schizochytrium spp., and Ulkenia spp.; diatoms, including, for example, (e.g., Bacillariophyceae): Achnanthes spp., Amphora spp., Caloneis spp., Camphylodiscus spp., Cymbella spp., Entomoneis spp., Gyrosigma spp., Melosira spp., Fragilaria spp., Cylindrotheca spp., Navicula spp., Nitzschia spp., Pleurosigma spp., Surirella spp., Chaetoceros muelleri, Cyclotella spp., and Phaeodactylum tricornutum; green algae (Chlorophyceae), including, for example, Chlamydomonas spp., Chlorella spp., Scenedesmus spp., Ankistrodesmus spp., Chlorococcum spp., Monoraphidium minutum, Nannochloris spp., Oocystis spp., Neochloris oleoabundans, Dunaliella primolecta, Botryococcus braunii, Tetraselmis suecica; blue-green algae (cyanobacteria or Cyanophyceae), including, for example, Synechococcus spp., Oscillatoria spp.; golden algae (Chrysophyceae), including, for example, Boekelovia spp., Isochrysis spp.; Prymnesiophyceae and Eustigmatophyceae, including, for example, Nannochloropsis spp.

“Autonomous” means that when delivered to plant cells, at least some MCs are transmitted through mitotic division to daughter cells and are episomal in the daughter plant cells, i.e., are not chromosomally integrated in the daughter plant cells. During the introduction into a cell of a MC, or during subsequent stages of the cell cycle, there may be chromosomal integration of some portion or all of the DNA derived from a MC in some cells. The MC is still characterized as autonomous despite the occurrence of such events if a plant, plant part or plant tissue can be regenerated that contains episomal descendants of the MC distributed throughout its parts, or if gametes or progeny can be derived from the plant that contain episomal descendants of the MC distributed through its parts.

A “centromere” is any DNA sequence that confers an ability to segregate to daughter cells through cell division. In one context, this sequence produces a segregation efficiency to daughter cells ranging from about 1% to about 100%, including to about 5%, 10%, 20%, 30%, 40%, 50%, 60%, 70%, 80%, 90% or about 95% of daughter cells. Variations in such a segregation efficiency can find important applications within the scope of the invention; for example, minichromosomes carrying centromeres that confer 100% stability can be maintained in all daughter cells without selection, while those that confer 1% stability can be temporarily introduced into a transgenic organism, but be eliminated when desired. A centromere can confer stable segregation of a nucleic acid sequence, including a recombinant construct comprising the centromere, through mitotic or meiotic divisions, including through both meiotic and meitotic divisions. An exogenously introduced centromere, such as on a MC, is not necessarily derived from the host organism, but has the ability to promote DNA segregation in the host cell.

“Centromere binding protein” (or “CAP”) refers to a polypeptide that binds with relatively high affinity and specificity to a centromere.

“Circular permutations” refer to variants of a sequence that begin at base n within the sequence, proceed to the end of the sequence, resume with base number one of the sequence, and proceed to base n−1. For this analysis, n can be any number less than or equal to the length of the sequence. For example, circular permutations of the sequence ABCD are: ABCD, BCDA, CDAB, and DABC.

“Consensus” refers to a nucleic acid sequence derived by comparing two or more related sequences. A consensus sequence defines both the conserved and variable sites between the sequences being compared.

“Crop” includes any plant or algae or portion of a plant or algae grown or harvested for commercial or beneficial purposes, including for the production of biofuels.

“Exogenous” when used in reference to a nucleic acid, for example, refers to any nucleic acid that has been introduced into a recipient cell, regardless of whether the same or similar nucleic acid is already present in such a cell. An “exogenous gene” can be a gene not normally found in the host genome in an identical context, or an extra copy of a host gene. The gene can be isolated from a different species than that of the host genome, or alternatively, isolated from the host genome but operably linked to one or more regulatory regions that differ from those found in the unaltered, native gene. The gene can also be synthesized in vitro.

“Functional” when referring to a MC, centromere, nucleic acid, or polypeptide, for example, retains a biological and/or an immunological activity of native or naturally-occurring chromosome, centromere, nucleic acid, or polypeptide, respectively. When used to describe an exogenouse nucleic acid carried on an MC, “functional” means that the exogenous nucleic acid can function in a detectable manner when the MC is within a cell, such as a plant cell; exemplary functions of the exogenous nucleic acid include transcription of the exogenous nucleic acid, expression of the exogenous nucleic acid, regulatory control of expression of other exogenous nucleic acids, recognition by a restriction enzyme or other endonuclease, ribozyme or recombinase; providing a substrate for DNA methylation, DNA glycolation or other DNA chemical modification; binding to proteins such as histones, helix-loop-helix proteins, zinc binding proteins, leucine zipper proteins, MADS box proteins, topoisomerases, helicases, transposases, TATA box binding proteins, viral protein, reverse transcriptases, or cohesins; providing an integration site for homologous recombination; providing an integration site for a transposon, T-DNA or retrovirus; providing a substrate for RNAi synthesis; priming of DNA replication; aptamer binding; or kinetochore binding. If multiple exogenous nucleic acids are present within the MC, the function of one or preferably more of the exogenous nucleic acids can be detected under suitable conditions permitting function.

“Higher eukaryote” means a multicellular eukaryote, typically characterized by its greater complex physiological mechanisms and relatively large size. Generally, complex organisms such as plants and animals are included. Higher eukaryotes are exemplified by monocot and dicot angiosperm species, gymnosperm species, fern species, plant tissue culture cells of these species, animal cells and algal cells.

“Linker” refers to a DNA molecule, generally up to 50 or 60 nucleotides. This fragment contains one, or more than one, restriction enzyme site.

“Lower eukaryote” refers to a eukaryote characterized by a comparatively simple physiology and composition and is usually unicellular. Examples of lower eukaryotes include flagellates, ciliates, and yeasts.

A “minichromosome” (“MC”) is a recombinant DNA construct including a centromere and is capable of being transmitted to daughter cells. A MC can remain separate from the host genome (as episomes) or can integrate into host chromosomes. The stability of this construct through cell division can range between from about 1% to about 100%, including about 5%, 10%, 20%, 30%, 40%, 50%, 60%, 70%, 80%, 90% and about 95%. The MC construct can be circular or linear. It can include elements such as one or more telomeres, origin of replication sequences, stuffer sequences, buffer sequences, chromatin packaging sequences, linkers and genes. The number of such sequences included is only limited by the physical size limitations of the construct itself. It can contain DNA derived from a natural centromere. The MC can also contain a synthetic centromere composed of tandem arrays of repeats of any sequence, either derived from a natural centromere, or of synthetic DNA. The MC can also contain DNA derived from multiple natural centromeres. The MC can be inherited through mitosis or meiosis, or through both meiosis and mitosis. The term “minichromosome” or “MC” specifically encompasses and includes the terms “artificial chromosome,” “plant artificial chromosomes,” “PLAC,” or “AC,” or engineered chromosomes or microchromosomes and all teachings relevant to a PLAC or plant artificial chromosome specifically apply to constructs within the meaning of the term MC.

“Operably linked” means a configuration in that a control sequence, e.g., a promoter sequence, directs transcription or translation of another sequence, for example a coding sequence. For example, a promoter sequence could be appropriately placed at a position relative to a coding sequence such that the control sequence directs the production of a polypeptide encoded by the coding sequence.

The term “plant,” as used herein, refers to any type of plant. Exemplary types of plants are listed below, but other types of plants will be known to those of skill in the art and could be used with the invention. Modified plants of the invention include, for example, dicots, gymnosperm, monocots, mosses, ferns, horsetails, club mosses, liver worts, homworts, red algae, brown algae, gametophytes and sporophytes of pteridophytes, and green algae.

A common class of plants exploited in agriculture are vegetable crops, including artichokes, kohlrabi, arugula, leeks, asparagus, lettuce (e.g., head, leaf, romaine), bok choy, malanga, broccoli, melons (e.g., muskmelon, watermelon, crenshaw, honeydew, cantaloupe), brussels sprouts, cabbage, cardoni, carrots, napa, cauliflower, okra, onions, celery, parsley, chick peas, parsnips, chicory, Chinese cabbage, peppers, collards, potatoes, cucumber plants (marrows, cucumbers), pumpkins, cucurbits, radishes, dry bulb onions, rutabaga, eggplant, salsify, escarole, shallots, endive, garlic, spinach, green onions, squash, greens, beet (sugar beet or fodder beet), sweet potatoes, swiss chard, horseradish, tomatoes, kale, turnips, or spices.

Other types of plants frequently finding commercial use include fruit and vine crops such as apples, grapes, apricots, cherries, nectarines, peaches, pears, plums, prunes, quince, almonds, chestnuts, filberts, pecans, pistachios, walnuts, citrus, blueberries, boysenberries, cranberries, currants, loganberries, raspberries, strawberries, blackberries, grapes, avocados, bananas, kiwi, persimmons, pomegranate, pineapple, tropical fruits, pomes, melon, mango, papaya, or lychee.

Modified wood and fiber or pulp plants of particular interest include, but are not limited to maple, oak, cherry, mahogany, poplar, aspen, birch, beech, spruce, fir, kenaf, pine, walnut, cedar, redwood, chestnut, acacia, bombax, alder, eucalyptus, catalpa, mulberry, persimmon, ash, honeylocust, sweetgum, privet, sycamore, magnolia, sourwood, cottonwood, mesquite, buckthorn, locust, willow, elderberry, teak, linden, bubing a, basswood or elm.

Modified flowers and ornamental plants of particular interest, include roses, petunias, pansy, peony, olive, begonias, violets, phlox, nasturtiums, irises, lilies, orchids, vinca, philodendron, poinscttias, opuntia, cyclamen, magnolia, dogwood, azalea, redbud, boxwood, Viburnum, maple, elderberry, hosta, agave, asters, sunflower, pansies, hibiscus, morning glory, alstromeria, zinnia, geranium, Prosopis, artemesia, clematis, delphinium, dianthus, gallium, coreopsis, iberis, lamium, poppy, lavender, leucophyllum, scdum, salvia, verbascum, digitalis, penstemon, savory, pythrethrum, or oenolhera. Modified nut-bearing trees of particular interest include, but are not limited to pecans, walnuts, macadamia nuts, hazelnuts, almonds, or pistachios, cashews, pignolas or chestnuts.

Many of the most widely grown plants are field crop plants such as evening primrose, meadow foam, corn (field, sweet, popcorn), hops, jojoba, peanuts, rice, safflower, small grains (barley, oats, rye, wheat, etc.), sorghum, tobacco, kapok, leguminous plants (beans, lentils, peas, soybeans), oil plants (rape, mustard, poppy, olives, sunflowers, coconut, castor oil plants, cocoa beans, groundnuts, oil palms), fibre plants (cotton, flax, hemp, jute), lauraceae (cinnamon, camphor), or plants such as coffee, sugarcane, cocoa, tea, or natural rubber plants.

Still other examples of plants include bedding plants such as flowers, cactus, succulents or ornamental plants, as well as trees such as forest (broad-leaved trees or evergreens, such as conifers), fruit, ornamental, or nut-bearing trees, as well as shrubs or other nursery stock.

Modified crop plants of particular interest in the present invention include soybean (Glycine max), cotton, canola (also known as rape), wheat, sunflower, sorghum, alfalfa, barley, safflower, millet, rice, tobacco, fruit and vegetable crops or turfgrasses. Exemplary cereals include maize, wheat, barley, oats, rye, millet, sorghum, rice triticale, secale, einkorn, spelt, emmer, teff, milo, flax, gramma grass, Tripsacum sp., or teosinte. Oil-producing plants include plant species that produce and store triacylglycerol in specific organs, primarily in seeds. Such species include soybean (Glycine max), rapesecd or canola (including Brassica napus, Brassica rapa or Brassica campestris), Brassica juncea, Brassica carinata, sunflower (Helianthus annuus), cotton (including Gossypium hirsutum), corn (Zea mays), cocoa (Theobroma cacao), safflower (Carthamus tinctorius), oil palm (Elaeis guineensis), coconut palm (Cocos nucifera), flax {Linum usitatissimum), castor (Ricinus communis) or peanut (Arachis hypogaea). “Cotton” includes species of the genus Gossypium, including the commercially important cottons, Gossypium hirsutum (Upland cotton), Gossypium herbaceum (Levant cotton), Gossypium arboreum (Tree cotton), and Gossypium barbadense (Pima cotton).

“Plant part” includes pollen, silk, endosperm, ovule, seed, embryo, pods, roots, cuttings, tubers, stems, stalks, fiber (lint), square, boll, fruit, berries, nuts, flowers, leaves, bark, wood, whole plant, plant cell, plant organ, epidermis, vascular tissue, protoplast, cell culture, crown, callus culture, petiole, petal, sepal, stamen, stigma, style, bud, meristem, cambium, cortex, pith, sheath, or any group of plant cells organized into a structural and functional unit. In one preferred embodiment, the exogenous nucleic acid is expressed in a specific location or tissue of a plant, for example, epidermis, vascular tissue, meristem, cambium, cortex, pith, leaf, sheath, flower, root or seed.

“Probe” is any biochemical reagent (usually tagged in some way for ease of identification), used to identify or isolate a gene, a gene product, a DNA segment or a protein.

“Pseudogene” refers to a non-functional copy of a protein-coding gene; pseudogenes found in the genomes of eukaryotic organisms are often inactivated by mutations and are thus presumed to be non-essential to that organism; pseudogenes of reverse transcriptase and other open reading frames found in retroelements are abundant in the centromeric regions of Arabidopsis and other organisms and are often present in complex clusters of related sequences.

“Recombination” refers to any genetic exchange that involves breaking and rejoining of DNA strands.

“Regulatory sequence” refers to any DNA sequence that influences the efficiency of transcription or translation of any gene when operably linked to that gene. Examples of regulatory sequences include promoters, enhancers and terminators.

A “repeated nucleotide sequence” refers to any nucleic acid sequence of at least 25 bp, present in a genome or a recombinant molecule, other than a telomere repeat, that occurs at least two or more times and that are at least 80% identical either in head to tail or head to head orientation either with or without intervening sequence between repeat units. Repeated nucleotide sequences can be shorter than 25 bp.

“Retroelement” or “retrotransposon” refers to a genetic element related to retroviruses that disperse through an RNA stage; the abundant retroelements present in plant genomes contain long terminal repeats (LTR retrotransposons) and encode a polyprotein gene that is processed into several proteins including a reverse transcriptase. Specific retroelements (complete or partial sequences (e.g., “retroelement-like sequence” and “retrotransposon-like sequence”) can be found in and around plant centromeres and can be present as dispersed copies or complex repeat clusters. Individual copies of retroelements can be truncated or contain mutations; intact retrolements are rarely encountered.

“Satellite DNA” refers to short DNA sequences (typically <1000 bp) present in a genome as multiple repeats, mostly arranged in a tandemly repeated fashion, as opposed to a dispersed fashion. Repetitive arrays of specific satellite repeats are abundant in the centromeres of many higher eukaryotic organisms.

A “screenable marker” is a gene whose presence results in an identifiable phenotype. This phenotype can be observable under standard conditions, altered conditions such as elevated temperature, or in the presence of certain chemicals used to detect the phenotype.

A “selectable marker” is a gene whose presence results in a clear phenotype, and most often a growth advantage for cells that contain the marker. This growth advantage can be present under standard conditions, altered conditions such as elevated temperature, or in the presence of certain chemicals such as herbicides or antibiotics. Examples of selectable markers include the thymidine kinase gene, the cellular adenine phosphoribosyltransferase gene and the dihydrylfolate reductase gene, hygromycin phosphotransferase genes, the bar gene and neomycin phosphotransferase genes, among others.

“Site-specific recombination” refers to any genetic exchange that involves breaking and rejoining of DNA strands at a specific DNA sequence.

“Stable” means that a MC can be transmitted to daughter cells over at least 8 mitotic generations. Some embodiments of MCs can be transmitted as functional, autonomous units for less than 8 mitotic generations, e.g., 1, 2, 3, 4, 5, 6, or 7. Preferred MCs can be transmitted over at least 8, 9, 10, 11, 12, 13, 14, 15, 16, 17, 18, 18, 19, 20, 21, 22, 23, 24, 25, 26, 27, 28, 29 or 30 mitotic generations, for example, through the regeneration or differentiation of an entire plant, and preferably are transmitted through meiotic division to gametes. Other preferred MCs can be further maintained in the zygote derived from such a gamete or in an embryo or endosperm derived from one or more such gametes. A “functional and stable” MC is one in that functional MCs can be detected after transmission of the MCs over at least 8 mitotic generations, or after inheritance through a meiotic division. During mitotic division, as occurs occasionally with native chromosomes, there can be some non-transmission of MCs; the MC can still be characterized as stable despite the occurrence of such events if an adchromosomal plant that contains descendants of the MC distributed throughout its parts can be regenerated from cells, cuttings, propagules, or cell cultures containing the MC, or if an adchromosomal plant can be identified in progeny of the plant containing the MC.

“Structural gene” is a sequence that codes for a polypeptide or RNA and includes 5′ and 3′ ends. The structural gene can be from the host into which the structural gene is transformed or from another species. A structural gene usually includes one or more regulatory sequences that modulate the expression of the structural gene, such as a promoter, terminator or enhancer. Structural genes often confer some useful phenotype upon an organism comprising the structural gene, for example, herbicide resistance. A structural gene can encode an RNA sequence that is not translated into a protein, for example a tRNA or rRNA gene.

“Synthetic,” when used in the context of a polynucleotide or polypeptide, refers to a molecule that is made using standard synthetic techniques, e.g., using an automated DNA or peptide synthesizer. Synthetic sequence can be a native sequence, or a modified sequence.

“Telomere” refers to a sequence capable of capping the ends of a chromosome, preventing degradation of the chromosome end, ensuring replication and preventing fusion to other chromosome sequences. Telomeres can include naturally occurring telomere sequences or synthetic sequences. Telomeres from one species can confer telomere activity in another species.

“Trait” refers either to the altered phenotype of interest or the nucleic acid that causes the altered phenotype of interest.

“Transformed,” “transgenic,” “modified,” and “recombinant” refer to a host organism such as a plant into which an exogenous or heterologous nucleic acid molecule has been introduced, and includes whole plants, meiocytes, seeds, zygotes, embryos, endosperm, or progeny of such plants that retain the exogenous or heterologous nucleic acid molecule but that have not themselves been subjected to the transformation process.

“Transmission efficiency” of a certain percent is calculated by measuring MC presence through one or more mitotic or meiotic generations. It is directly measured as the ratio (expressed as a percentage) of the daughter cells or plants demonstrating presence of the MC to parental cells or plants demonstrating presence of the MC. Presence of the MC in parental and daughter cells is demonstrated with assays that detect the presence of an exogenous nucleic acid carried on the MC. Exemplary assays can be the detection of a screenable marker (e.g., presence of a fluorescent protein or any gene whose expression results in an observable phenotype), a selectable marker, or PCR amplification of any exogenous nucleic acid carried on the MC.

An “isolated” or “purified” protein or biologically active portion thereof is substantially free of cellular material or other contaminating proteins from the cell or tissue source from that the isolated protein is derived, or substantially free from chemical precursors or other chemicals when chemically synthesized. “Substantially free of cellular material” means, for example, preparations of an isolated protein having less than about 30% (by dry weight) of contaminating protein, less than about 20%, 10%, or 5% of contaminating protein.

A “native sequence polypeptide” comprises a polypeptide having the same amino acid sequence as the corresponding polypeptide derived from nature. Such native sequence polypeptides can be isolated from nature or can be produced by recombinant or synthetic means. The term “native sequence polypeptide” specifically encompasses naturally-occurring truncated or secreted forms of the specific polypeptide (e.g., an extracellular domain sequence), naturally-occurring variant forms (e.g., alternatively spliced forms) and naturally-occurring allelic variants of the polypeptide.

A “polypeptide variant” means an active polypeptide having at least about 70% amino acid sequence identity with a full-length native sequence polypeptide sequence or any other fragment of a full-length polypeptide. Such polypeptide variants include, for instance, polypeptides wherein one or more amino acid residues are added, or deleted, at the N- or C-terminus of the full-length native amino acid sequence. Ordinarily, a polypeptide variant will have at least about 70% amino acid sequence identity, at least about 80%, 81%, 82%, 83%, 84%, 85%, 86%, 87%, 88%, 89%, 90%, 91%, 92%, 93%, 94%, 95%, 96%, 97%, 98%, or 99% amino acid sequence identity with a full-length native sequence polypeptide sequence, a polypeptide sequence lacking the signal peptide as disclosed herein, an extracellular domain of a polypeptide, with or without the signal peptide, as disclosed herein or any other specifically defined fragment of a full-length polypeptide sequence as disclosed herein. Ordinarily, variant polypeptides are at least about 10 amino acids, or 20, 30, 40, 50, 60, 70, 80, 90, 100, 150, 200 250, or 300 or more amino acids in length.

“Percent (%) amino acid sequence identity” with respect to a polypeptide sequence is defined as the percentage of amino acid residues in a candidate sequence that is identical with the amino acid residues in the specific polypeptide sequence, after aligning the sequences and introducing gaps, if necessary, to achieve the maximum percent sequence id entity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

The % amino acid sequence identity of a given amino acid sequence A to, with, or against a given amino acid sequence B (that can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment algorithm in the alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

A “polynucleotide” is a nucleic acid polymer of ribonucleic acid (RNA), deoxyribonucleic acid (DNA), modified RNA or DNA, or RNA or DNA mimetics (such as PNA5), and derivatives thereof, and homologues thereof. Thus, polynucleotides include polymers composed of naturally occurring nucleobases, sugars and covalent inter-nucleoside (backbone) linkages as well as polymers having non-naturally-occurring portions that function similarly. Such modified or substituted nucleic acid polymers are well known in the art and for the purposes of the present invention, are referred to as “analogues.” Oligonucleotides are generally short polynucleotides from about 10 to up to about 160 or 200 nucleotides.

A “variant polynucleotide” or a “variant nucleic acid sequence” means a polynucleotide having at least about 60% nucleic acid sequence identity, more at least about 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% nucleic acid sequence identity and yet more at least about 99% nucleic acid sequence identity with the nucleic acid sequence of a sequence of interest. Variants do not encompass the native nucleotide sequence.

Ordinarily, variant polynucleotides are at least about 8 nucleotides in length, often at least about 9, 10, 11, 12, 13, 14, 15, 16, 17, 18, 19, 20, 21, 22, 23, 24, 25, 26, 27, 28, 29 30, 35, 40, 45, 50, 55, 60 nucleotides in length, or even about 75-200 nucleotides in length, or more.

“Percent (%) nucleic acid sequence identity” with respect to nucleic acid sequences is defined as the percentage of nucleotides in a candidate sequence that is identical with the nucleotides in the sequence of interest, after aligning the sequences and introducing gaps, if necessary, to achieve the maximum percent sequence identity. Alignment for purposes of determining % nucleic acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

When nucleotide sequences are aligned, the % nucleic acid sequence identity of a given nucleic acid sequence C to, with, or against a given nucleic acid sequence D (that can alternatively be phrased as a given nucleic acid sequence C that has or comprises a certain % nucleic acid sequence identity to, with, or against a given nucleic acid sequence D) can be calculated as follows:

% nucleic acid sequence identity=W/Z·100

where

W is the number of nucleotides cored as identical matches by the sequence alignment program's or algorithm's alignment of C and D

and

Z is the total number of nucleotides in D.

When the length of nucleic acid sequence C is not equal to the length of nucleic acid sequence D, the % nucleic acid sequence identity of C to D will not equal the % nucleic acid sequence identity of D to C.

“Consisting essentially of a polynucleotide having a % sequence identity” means that the polynucleotide does not substantially differ in length, but can differ substantially in sequence. Thus, a polynucleotide “A” consisting essentially of a polynucleotide having at least 80% sequence identity to a known sequence “B” of 100 nucleotides means that polynucleotide “A” is about 100 nts long, but up to 20 nts can vary from the “B” sequence. The polynucleotide sequence in question can be longer or shorter due to modification of the termini, such as, for example, the addition of 1-15 nucleotides to produce specific types of probes, primers and other molecular tools, etc., such as the case of when substantially non-identical sequences are added to create intended secondary structures. Such non-identical nucleotides are not considered in the calculation of sequence identity when the sequence is modified by “consisting essentially of.”

“Hybridizes under low stringency, medium stringency, and high stringency conditions” describes conditions for hybridization and washing. Hybridization is a well-known technique (Ausubel 1987). Low stringency hybridization conditions means, for example, hybridization in 6× sodium chloride/sodium citrate (SSC) at about 45° C., followed by two washes in 0.5×SSC, 0.1% SDS, at least at 50° C.; medium stringency hybridization conditions means, for example, hybridization in 6×SSC at about 45° C., followed by one or more washes in 0.2×SSC, 0.1%) SDS at 55° C.; and high stringency hybridization conditions means, for example, hybridization in 6×SSC at about 45° C., followed by one or more washes in 0.2×SSC, 0.1% SDS at 65° C. Another non limiting example of stringent hybridization conditions are hybridization in a high salt buffer comprising 6×SSC, 50 mM Tris HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. Another non limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Another non limiting example of low stringency hybridization conditions are hybridization in 35% formamide, 5×SSC, 50 mM Tris HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris HCl (pH 7.4), 5 mM EDTA, and 0.1% SDS at 50° C. Other conditions of low stringency that may be used are well known in the art (e.g., as employed for cross species hybridizations).

“Antibody” is used in the broadest sense and specifically covers, for example, single anti-CAP monoclonal antibodies (including agonist, antagonist, and neutralizing antibodies), anti-CAP antibody compositions with polyepitopic specificity, single chain anti-CAP antibodies, and fragments of anti-CAP antibodies (see below). “Monoclonal antibody” refers to an antibody obtained from a population of substantially homogeneous antibodies, i.e., the individual antibodies comprising the population are identical except for possible naturally-occurring mutations that can be present in minor amounts.

“Epitope tagged” refers to a chimeric polypeptide comprising a polypeptide fused to a “tag polypeptide.” The tag polypeptide has enough residues to provide an epitope against that an antibody can be made, yet is short enough such that it does not interfere with activity of the polypeptide to that it is fused. Preferably, the tag polypeptide is fairly unique so that the antibody does not substantially cross-react with other epitopes. Suitable tag polypeptides generally have at least six amino acid residues and usually between about 8 and 50 amino acid residues.

“Immunoadhesin” designates antibody-like molecules that combine the binding specificity of a heterologous protein (an “adhesin”) with the effector functions of immunoglobulin constant domains. Structurally, the immunoadhesins comprise a fusion of an amino acid sequence with the desired binding specificity that is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin can be obtained from any immunoglobulin, such as IgG-1, IgG-2, IgG-3, or IgG-4 subtypes, IgA (including IgA-1 and IgA-2), IgE, IgD or IgM.

### III. Making and Using the Invention

### A. Selected Embodiments

The following embodiments are not meant to limit the invention in any way.

The invention relates to centromeres identified using the disclosed methods, and recombinant nucleic acid molecules that include centromere sequences and variants thereof. The invention includes minichromosomes that include centromeres identified using the methods of the inventions.

In one aspect, the invention includes methods of identifying a centromere sequence that include precipitating protein-DNA complexes from chromatin isolated from a cell using an antibody to, or molecules that bind specifically to, centromere-associated proteins; isolating nucleic acid molecules from the precipitated protein-DNA complexes; and sequencing the isolated nucleic acid molecules to identify a centromere sequence or used as probes to identify clones in libraries of genomic DNA. In some embodiments the nucleic acid molecules isolated from immunoprecipitated protein-DNA complexes are amplified prior to sequencing.

In addition to ChIP-based approaches, other embodiments used methods that depend on a CAP, but do not require precipitation. One alternative to ChIP is DNA adenine methyltransferase identification (DamID) (van Steensel and Henikoff 2000). In this method, the protein of interest (e.g. CenH3) is fused to the bacterial DNA methyltransferase Dam which catalyses the addition of a methyl group to adenine nucleotides. The fusion protein is then expressed in the cell of interest and will methylate adenines wherever the protein binds DNA. Since adenines are not normally methylated in eukaryotes, the DNA binding targets of the protein of interest can be isolated by virtue of their methylation status (for example by using restriction enzymes that are sensitive to Dam methylation followed by gel electrophoresis). DamID is an attractive alternative to ChIP since it does not require the production of an antibody to the protein of interest. Another alternative to ChIP is the commercial product offered by Promega called HaloTag™ (Urh, Hartzell et al. 2008). In this method, the protein of interest (e.g. CenH3) is fused to the HaloTag protein which has the ability to tightly bind chloroalkane resins. The fusion protein is expressed in the cell type of interest where it can bind its target DNA sequence. Chromatin in extracted from the cell, crosslinked and passed over the resin. Only DNA that is bound by the HaloTag fusion is retained on the column. The crosslink is then reversed and the DNA can be examined. Like DamID, HaloTagging has the advantage of not requiring an antibody to the protein of interest. A third alternative technology to ChIP is the electrophoretic mobility shift assay (EMSA) (Garner and Revzin 1981). In this approach, target DNA is labeled and incubated with the purified protein of interest (e.g. CenH3). The reaction is then subject to gel electrophoresis and protein-DNA interactions are detected as mobility shifts of the labeled DNA compared to control samples not bound by the protein. Shifted DNA can be extracted from the gel and examined. EMSA has the advantage of not requiring an antibody to the protein of interest nor requiring that the protein be made into a fusion. Yet another alternative to ChIP is Southwestern blotting (Siu, Lee et al. 2008). In this method the protein of interest (e.g. CenH3) is electrophoresed, typically on a polyacrylamide gel (i.e. SDS-PAGE or native PAGE), and transferred to a membrane. The membrane is then incubated with labeled DNA and the protein DNA interaction is visualized (e.g. by autoradiography for radiolabeled DNA). Modifications of this procedure also include incubating the gel directly with the labeled DNA rather than transferring the proteins to a membrane. The interacting DNA can then be recovered and analyzed. Southwestern blotting has the advantage of not needing an antibody to the protein of interest and not requiring fusions to be made—furthermore, because the gel electrophoresis provides molecular weight information the protein does not necessarily need to be fully purified.

In all embodiments, sequence identity to known centromere sequences is not normally used as a basis to establish new centromere sequences. For example, the methods of the invention do not include hybridization of nucleic acid molecules isolated from precipitated protein-DNA complexes to confirmed or putative centromere sequences or clones, such as sequences having a repeated sequence motif, and do not include comparison of sequences obtained by sequencing of affinity-captured products to sequences previously identified as putative centromere sequences or centromere-proximal sequences.

A high frequency of occurrence of a sequence in a population of sequences isolated using chromatin precipitation correlates with the likelihood of that sequence containing centromere sequence.

One aspect of the invention is related to organisms, such as alga or fungi, containing functional, stable, autonomous MCs, preferably carrying one or more exogenous nucleic acids. Such organisms carrying MCs are contrasted to transgenic organisms that have altered genomes by chromosomal integration of an exogenous nucleic acid. Expression of the exogenous nucleic acid results in an altered phenotype of the organism. The invention provides for MCs comprising at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 150, 250, 500, 1000 or more exogenous nucleic acids.

The MC can be transmitted to subsequent generations of viable daughter cells during mitotic cell division with a transmission efficiency of at least 1%, 5%, 10%, 20%, 30%, 40%, 50%, 60%, 70%, 80%, 85%, 90%, 95%, 96%, 97%, 98%, or 99%. The MC is transmitted to viable gametes during meiotic cell division with a transmission efficiency of at least 1%, 5%, 10%, 20%, 30%, 40%, 50%, 60%, 70%, 80%, 85%, 90%, 95% o, 96%, 97%, 98%, or 99% when more than one copy of the MC is present in the gamete mother cells of the plant. The MC is transmitted to viable gametes during meiotic cell division with a transmission frequency of at least 1%, 5%, 10%, 20%, 30%, 40%, 45%, 46%, 47%, 48%, or 49% when one copy of the MC is present in the gamete mother cells of the organisms and meiosis produces four viable products (e.g. typical plant male meiosis) When meiosis produces fewer than four viable products (e.g. typical plant female meiosis) a phenomenon called meiotic drive can cause the preferential segregation of particular chromosomes into the viable product resulting in higher than expected transmission frequencies of monoosmes through meiosis including at least 51%, 60%, 70%, 80%, 90% 95%, 96%, 97%, 98%, or 99%. For production of seeds via sexual reproduction or by apomyxis, the MC can be transferred into at least 1%, 5%, 10%, 20%, 25%, 30%, 40%, 50%, 60%, 70%, 80%, 85%, 90%, 95%, 96%, 97%, 98%, or 99% of viable embryos when cells of the plant contain more than one copy of the MC. For sexual seed production or apomyxitic seed production from plants with one MC per cell, the MC can be transferred into at least 1%, 5%, 10%, 20%, 30%, 40%, 50%, 60%, 70%, 71%, 72%, 73%, 74%, 75% of viable embryos.

A MC that comprises an exogenous selectable trait or exogenous selectable marker can be used to increase the frequency in subsequent generations of cells, tissues, gametes, embryos, endosperm, seeds, plants or progeny. For example, the frequency of transmission of MCs can be significantly increased after mitosis or meiosis by applying a selection that favors the survival of MC-carrying cells.

Transmission efficiency can be measured as the percentage of progeny cells or organisms that carry the MC by one of several assays, including detecting expression of a reporter gene (e.g., a gene encoding a fluorescent protein), PCR detection of a sequence that is carried by the MC, RT-PCR detection of a gene transcript for a gene carried on the MC, Western analysis of a protein produced by a gene carried on the MC, Southern analysis of the DNA (either in total or a portion thereof) carried by the MC, fluorescence in situ hybridization (FISH) or in situ localization by repressor binding. Efficient transmission as measured by some benchmark percentage indicates the degree to which the MC is stable through the mitotic and meiotic cycles. Plants of the invention can also contain chromosomally integrated exogenous nucleic acid in addition to the autonomous MCs. The MC-containing organisms can include those that have chromosomal integration of some portion of the MC (e.g., exogenous nucleic acid or centromere sequence) in some or all cells of the organism.

Exemplary MCs of the invention are contemplated to be of a size 2000 kb or less. Other exemplary sizes of MCs include less than or equal to, e.g., 1500 kb, 1000 kb, 900 kb, 800 kb, 700 kb, 600 kb, 500 kb, 450 kb, 400 kb, 350 kb, 300 kb, 250 kb, 200 kb, 150 kb, 100 kb, 90 kb, 80 kb, 70, kb, 60 kb, or 40 kb. However, the size of MCs are typically limited by the technologies that are used to handle such large molecules in the lab.

Novel centromere compositions as characterized by sequence content, size, spatial arrangement of sequence motifs, or other parameters. It can be advantageous to use minimal size of centromeric sequence in MC construction. Exemplary sizes include a centromeric nucleic acid insert derived from a portion of genomic DNA, that is less than or equal to 1000 kb, 900 kb, 800 kb, 700 kb, 600 kb, 500 kb, 400 kb, 300 kb, 200 kb, 150 kb, 100 kb, 95 kb, 90 kb, 85 kb, 80 kb, 75 kb, 70 kb, 65 kb, 60 kb, 55 kb, 50 kb, 45 kb, 40 kb, 35 kb, 30 kb, 25 kb, 20 kb, 15 kb, 10 kb, 5 kb, 4 kb, 3 kb, 2 kb, or 1 kb.

### B. Composition of MCs and MC Construction

The MCs of the present invention can contain a variety of elements, including: (1) sequences that function as centromeres; (2) one or more exogenous nucleic acids; (3) sequences that function as an origin of replication, that can be included in the region that functions as centromere; (4) optionally, a bacterial plasmid backbone for propagation of the plasmid in bacteria, though this element may be designed to be removed prior to delivery to a target cell; (5) sequences that function as telomeres (particularly if the MC is linear); (6) optionally, additional “stuffer DNA” sequences that serve to separate the various components on the MC from each other; (7) optionally, “buffer” sequences such as MARs or SARs; (8) optionally, marker sequences of any origin; (9) optionally, sequences that serve as recombination sites; and (10) optionally, “chromatin packaging sequences” such as cohesion and condensing binding sites.

### C. Novel Centromere Compositions

The centromere in the MCs of the present invention, identified using the methods of the invention, can comprise novel repeating centromeric sequences; or, alternatively, the centromere of the MCs of the present invention comprise “point” centromeres or structural motifs that are “bent DNA.”

MC Sequence Content and Structure

Exogneous genes can be modified to accommodate the host organism's codon usage if necessary, to insert preferred motifs near the translation initiation ATG codon, to remove sequences recognized in plants as 5′ or 3′ splice sites, or to better reflect plant GC/AT content.

Each exogenous nucleic acid or gene can include a promoter, a coding region and a terminator sequence, that can be separated from each other by restriction endonuclease sites or recombination sites or both. Genes can also include introns, native or artificial.

The coding regions of the genes can encode any protein, including visible marker genes (for example, fluorescent protein genes, other genes conferring a visible phenotype), other screenable or selectable marker genes (for example, conferring resistance to antibiotics, herbicides or other toxic compounds, or encoding a protein that confers a growth advantage to the cell expressing the protein) or genes that confer some commercial or agronomic value to the host organism. Multiple genes can be placed on the same MC vector. The genes can be separated from each other by restriction endonuclease sites, homing endonuclease sites, recombination sites or any combinations thereof. Any number of genes can be present. Genes on a MC can be in any orientation with respect to one another and with respect to the other elements of the MC (e.g. the centromere).

The MC vector can also contain a bacterial plasmid backbone for propagation of the plasmid in bacteria such as E. coli, A. tumefaciens, or A. rhizogenes. The backbone can include one or several antibiotic-resistance genes conferring resistance to a specific antibiotic to the bacterial cell in that the plasmid is present. The backbone can also be designed so that it can be excised from the MC prior to delivery to a plant cell. The use of flanking restriction enzyme sites or flanking site-specific recombination sites are both useful for constructing a removable backbone.

The MC vector can also contain telomeres, which are well-known in the art.

Additionally, the MC vector can contain “stuffer DNA” sequences that serve to separate the various components on the MC. Stuffer DNA can be of any origin and can be synthetic or native, can be any convenient length, and can be repetitive in sequence, with unit repeats from 10 bp to 1 Mb. Examples of repetitive sequences that can be used as stuffer DNAs include rDNA, satellite repeats, retroelements, transposons, pseudogenes, transcribed genes, microsatellites, tDNA genes, and short sequence repeats. Stuffer sequences can also include DNA that can form boundary domains, such as scaffold attachment regions (SARs) or matrix attachment regions (MARs).

In one embodiment of the invention, the MC has a circular structure without telomeres. In another embodiment, the MC has a circular structure with telomeres. In a third embodiment, the MC has a linear structure with telomeres.

Various structural configurations of the MC elements are possible. A centromere can be placed on a MC either between genes or outside a cluster of genes next to a telomere. Stuffer DNAs can be combined with these configurations including stuffer sequences placed inside the telomeres, around the centromere between genes or any combination thereof. Thus, a large number of alternative MC structures are possible, depending on the relative placement of centromere DNA, genes, stuffer DNAs, bacterial sequences, telomeres, and other sequences. Such variations in architecture are possible both for linear and for circular MCs.

Exemplary Centromere Components

In one embodiment, the centromere contains n copies of a repeated nucleotide sequence, identified using the methods of the invention, wherein n is at least 2. In another embodiment, the centromere contains n copies of interdigitated repeats. An interdigitated repeat is a DNA sequence that consists of two distinct repetitive elements that combine to create a unique permutation. Potentially any number of repeat copies capable of physically being placed on the recombinant construct could be included on the construct, including about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 120, 140, 150, 200, 300, 400, 500, 750, 1,000, 1,500, 2,000, 3,000, 5,000, 7,500, 10,000, 20,000, 30,000, 40,000, 50,000, 60,000, 70,000, 80,000, 90,000 and about 100,000, including all ranges in-between such copy numbers. Moreover, the copies can vary from each other, such as is commonly observed in naturally occurring centromeres. The length of the repeat can vary, but usually range from about 20 bp to about 360 bp, from about 20 bp to about 250 bp, from about 50 bp to about 225 bp, from about 75 bp to about 210 bp, such as a 92 bp repeat and a 97 bp repeat, from about 100 bp to about 205 bp, from about 125 bp to about 200 bp, from about 150 bp to about 195 bp, from about 160 bp to about 190 and from about 170 bp to about 185 bp including about 180 bp. The length of the repeat can also be about 100 to 210 bp; such as 100, 194, and 210 bp. The length of the repeat can also include larger sequences, from about 300 bp to about 10 kb, from about 1 kb to 9 kb, from about 2 kb to about 8 kb, from about 3 kb to about 7 kb, from about 4 kb to about 8 kb, including, for example, 982 bp, 2836 bp, 5788 bp and 8308 bp.

Modification of Centromeres Isolated from Native Genome

Modification and changes can be made in the centromeric DNA segments of the current invention and still obtain a functional molecule with desirable characteristics. The following is a discussion based upon changing the nucleic acids of a centromere to create an equivalent, or even an improved, second generation molecule.

Mutated centromeric sequences can be useful for increasing the utility of the centromere. The function of the centromeres of the current invention can be based in part or in whole upon the secondary structure of the DNA sequences of the centromere, modification of the DNA with methyl groups or other adducts, and/or the proteins that interact with the centromere. By changing the DNA sequence of the centromere, one can alter the affinity of one or more centromere-associated protein(s) for the centromere and/or the secondary structure or modification of the centromeric sequences, thereby changing the activity of the centromere. Alternatively, changes can be made in the centromeres that do not affect the activity of the centromere. Changes in the centromeric sequences that reduce the size of the DNA segment needed to confer centromere activity are particularly useful, as are changes that increase the fidelity with that the centromere is transmitted during mitosis and meiosis.

Examples of Cargo Delivered by MCs

Of particular interest in the present invention are exogenous nucleic acids that when introduced into an organism, alter the phenotype of the organism or organism part. Such exogenous nucleic acids can be delivered on MCs. Exemplary exogenous nucleic acids encode polypeptides involved in one or more important biological properties in the organism. Other exemplary exogenous nucleic acids alter expression of exogenous or endogenous genes, either increasing or decreasing expression, optionally in response to a specific signal or stimulus. Other exemplary exogenous nucleic acids encode polypeptides that produce a trait in the organism that is not native to the organism.

One of the major purposes of transformation of organisms is to add some commercially desirable, important traits to the plant. Such traits include, for example, herbicide resistance or tolerance (especially in crop plants); insect (pest) resistance or tolerance; nematode resistance, disease resistance or tolerance (viral, bacterial, fungal, or other pathogens); stress tolerance and/or resistance, as exemplified by resistance or tolerance to drought, heat, chilling, freezing, excessive moisture, salt stress, mechanical stress, extreme acidity, alkalinity, toxins, UV light, ionizing radiation or oxidative stress; increased yields, whether in quantity or quality; enhanced or altered nutrient acquisition and enhanced or altered metabolic efficiency; enhanced or altered nutritional content (including altered gossypol levels) and makeup of plant tissues used for food, feed, fiber or processing; physical appearance; male sterility; drydown; standability; prolificacy; altered geographical range; altered day-length tolerance; starch quantity and quality; oil quantity and quality; protein quality and quantity; amino acid composition; modified chemical production; altered pharmaceutical or nutraceutical properties; altered bioremediation properties; increased biomass; altered growth rate; altered fitness; altered biodegradability; altered CO₂ fixation; presence of bioindicator activity; altered digestibility by humans or animals; altered allergenicity; altered mating characteristics; altered gene flow patterns; improved environmental impact; altered nitrogen fixation capability; the production of a pharmaceutically active protein; the production of a small molecule with medicinal properties; the production of a chemical including those with industrial utility; the production of fibers including those used in making clothing, towels, bedding, wall coverings, upholstery, draperies, textiles, yarn, thread, wicks, string, paper, medical bandages, cotton balls, cotton batting, cotton swabs, cotton wool, gauze, tampons and other feminine hygiene products, cellulose products (e.g. rayon, plastics, photographic film, and cellophane), tarps and other industrial materials; the production of nutraceuticals, food additives, carbohydrates, RNAs, lipids, fuels, dyes, pigments, vitamins, scents, flavors, vaccines, antibodies, hormones, and the like; and alterations in plant architecture or development, including changes in developmental timing, photosynthesis, signal transduction, cell growth, reproduction, or differentiation. Additionally one could create a library of an entire genome from any organism or organelle including mammals, plants, microbes, fungi, or bacteria, represented on MCs.

A modified organism can exhibit increased or decreased expression or accumulation of a product that can be a natural product of the organisms or a new or altered product. Examples of products include enzymes, RNA molecules, nutritional proteins, structural proteins, amino acids, lipids, fatty acids, polysaccharides, sugars, alcohols, alkaloids, carotenoids, propanoids, phenylpropanoids, terpenoids, steroids, flavonoids, phenolics, anthocyanins, pigments, vitamins or plant hormones. The modified organism can have enhanced or diminished requirements for light, water, nitrogen, nutrients, or trace elements. Modified organisms, such as plants and alga, can also have an enhanced ability to capture or fix nitrogen from the environment. Modifications can include overexpression, underexpression, antisense modulation, sense suppression, inducible expression, inducible repression, or inducible modulation of a gene.

### Methods of Identifying and Isolating Centromeres

Any CAP can be used in the methods of the invention to identify centromere sequences; however, CenH3 and CenpB (and their homologues throughout different genera) are preferred. Table 1 lists examples of CAPs and other centromere-associated proteins that can be used in the methods of the invention.

It should be noted that in addition to the CAPs listed in Table 1, any other protein that associates directly or indirectly with a chromosome's centromere or kinetochore can be used.

In one embodiment, a CAP of interest is generated in vitro, such as subcloning a polynucleotide encoding the CAP of interest and expressing it in a suitable host, such as E. coli, yeast, mammalian cells, insect cells, plant cells or algal cells and then purifying the produced CAP. Such purification can be facilitated by affinity tagging the CAP.

In another embodiment, a molecule that specifically binds to the target CAP is used, such as an anti-CAP antibody. Such antibodies can easily be raised in a host of species, including rabbit, cow, goat, chicken, mouse and rat, and be prepared as polyclonal or monoclonal. The antigen can be whole CAP (whether isolated from cells as native protein, synthesized in vito, or produced recombinantly), or small peptides of the target CAP that are preferably unique to the CAP, at least in the systems to be assayed. The antibodies can be affinity purified before use, processed into useful fragments, or tagged.

For methods depending on chromatin isolation (fragmented are not), the methods of the invention can use chromatin isolated from any eukaryotic organism, including plants, algae, and protists. Furthermore, chromatin from fungi can be used, including chytrids, blastocladiomycetes, neocallimastigomycetes, zgomycetes, trichomycetes, glomeromycotes, ascomycetes, or basidiomycetes. Examples of protists include members of the Labyrinthulomycota, water molds, slime molds (mxomycota), and protozoans.

Chromatin isolation and chromatin immunoprecipitation can be performed under a variety of conditions; the technique and its variants have been thoroughly reviewed by (Collas 2010). Some examples using the technique are disclosed in, for example, U.S. Pat. No. 6,410,243 and (Wang, Tang et al. 2002; Casas-Mollano, van Dijk et al. 2007). Buffers, detergents, salts, pH, cross-linking (if used) and fragmentation conditions can be adjusted as need to increase specificity.

Once a selected CAP or anti-CAP reagent, is in hand, there are many ways in which such a screen or purification could be done, including but not limited to:

interaction of CAP with random genomic sequences or with pooled, cloned, or otherwise selected DNA sequences in solution, followed by immunoprecipitation (ChIP method) and cloning of the precipitated sequences and their characterization by sequencing, or use of immunoprecipitated sequences as probes for blots or genomic libraries; by immobilization of selected DNA sequences (either purified or cloned, single or pooled) and use of the CAP as a protein probe to determine which DNA sequences bind CAP. Isolation or identification of the desired sequences, after binding CAP, could occur by use of a CAP-specific antiserum, or by epitope tagging of CAP prior to expression and purification, and detection with an antibody or antiserum specific to the epitope tag. These methods result in the identification of sequences of any length, including long (>25 kb) fragments of centromere DNA or other types of genomic DNA cloned into vectors capable of carrying large-inserts, that bind CAP and therefore are likely to have de novo centromere function.

If chromatin is being used a target from which to isolate CAP-binding sequences, chromatin fragmentation may be desired. Such fragmentation can be done during chromatin isolation, during the ChIP procedure, or even after isolation of CAP-nucleic acid complexes. Chromatin can be fragmented mechanically, chemically, or enzymatically. Chromatin can be fragmented by physical (mechanical) or chemical means, for example, by sonicating, shearing, or enzymatically digestion or chemical cleavage of DNA.

Once CAP-nucleic acid complexes are isolated, the nucleic acids can be sequenced or used as probes to identify subclones in genomic libraries. For sequencing, techniques that allow for the sequencing of a population of molecules are desirable, such as solid phase sequencing. The sequencing targets can be amplified before sequencing, as is well known to one of skill in the art.

To identify centromere sequences of the population of nucleic acid molecules isolated from CAP-nucleic acid complexes, sequences of a large number of the individual nucleic acids are determined, and a baseline frequency of the occurrence of a sequence is determined by looking for peaks of high coverage that may represent centromere sequences. Averaging of sequence coverage may be done across entire chromosomes if the sequence of the genome is available. While the presence of repeat sequences is characteristic of many higher eukaryotes, the possibility of point centromeres should also be kept in mind. An alternative to this approach is to group candidate centromere sequences by homology and to use representatives from each homology group as probes for fluorescence in situ hybridization (FISH) experiments using spread chromosomes from the appropriate species. In this approach centromere sequences should co-localize with physical features corresponding to the centromere such as the primary constriction on metaphase chromosome.

### E. Constructing MCs

MCS of the present invention minimally includes a centromere for conferring stable heritability and an origin of replication or “autonomous replication sequence” (ARS) allowing for continuing synthesis of the MC, which in some cases may be included in the centromere sequences. A MC may optionally also contain any of a variety of elements, including one or more exogenous nucleic acids, a bacterial or yeast plasmid backbone for propagation of the plasmid in bacteria; sequences that function as telomeres in the host organism, where the MC is not configured as a circular molecule, cloning sites; such as restriction enzyme recognition sites or sequences that serve as recombination sites; and “chromatin packaging sequences” such as cohesion and condensing binding sites or matrix.

In one embodiment, MCs can be constructed using site-specific recombination sequences (for example those recognized by the bacteriophage P1 Cre recombinase, or the bacteriophage lambda integrase, or similar recombination enzymes). A compatible recombination site, or a pair of such sites, is present on both the centromere containing DNA clones and the donor DNA clones. Incubation of the donor clone and the centromere clone in the presence of the recombinase enzyme causes strand exchange to occur between the recombination sites in the two plasmids; the resulting MCs contain centromere sequences as well as MC vector sequences. The DNA molecules formed in such recombination reactions is introduced into E. coli, other bacteria, yeast or plant cells by common methods in the field including, heat shock, chemical transformation, electroporation, particle bombardment, whiskers, or other transformation methods followed by selection for marker genes, including chemical, enzymatic, or color markers present on either parental plasmid, allowing for the selection of transformants harboring MCs.

### F. Methods of Detecting and Characterizing MCs in Cells or of Scoring MC Performance in Cells

Non-Selective MC Mitotic Inheritance Assays

The following assays can distinguish autonomous events from integrated events.

Assay #1: Transient Assay

MCs are tested for their ability to become established as chromosomes and their ability to be inherited in mitotic cell divisions. MCs are delivered to cells. The cells used can be at various stages of growth. The MC is then assessed over the course of several cell divisions, by tracking the presence of a screenable marker, e.g., a visible marker gene such as one encoding a fluorescent protein. Following initial delivery into many single cells and several cell divisions, single transformed cells divide to form clusters of MC-containing cells if the MC is inherited well.

Assay #2: Non-Lineage Based Inheritance Assays on Modified Transformed Cells

MC inheritance is assessed on modified cell by following the presence of the MC over the course of multiple cell divisions. An initial population of MC containing cells is assayed for the presence of the MC, by the presence of a marker gene, such as a gene encoding a fluorescent protein, a colored protein, a protein assayable by histochemical assay, or a gene affecting cell morphology. All nuclei are stained with a DNA-specific dye such as DAPI, Hoechst 33258, OliGreen, Giemsa YOYO, or TOTO, allowing a determination of the number of cells that do not contain the MC. After the initial determination of the percent of cells carrying the MC, the cells are allowed to divide over the course of several cell divisions. The number of cell divisions, n, is determined by an appropriate method, such as monitoring the change in total weight of cells, monitoring the change in volume of the cells, or directly counting cells in an aliquot of the culture. After a number of cell divisions, the population of cells is again assayed for the presence of the MC. The loss rate per generation is calculated by the equation (I):

Loss rate per generation=1−(F/1)^{1/n} (I)

Assay #3: Lineage-Based Inheritance Assays on Modified Cells

MC inheritance is assessed on modified cell lines by following the presence of the MC over the course of multiple cell divisions. In cell types that allow for tracking of cell lineage, such as plant root cell files, trichomes, and leaf stomata guard cells, MC loss per generation does not need to be determined statistically over a population, it can be discerned directly through successive cell divisions. In other manifestations of this method, cell lineage can be discerned from cell position, or methods including but not limited to the use of histological lineage tracing dyes, and the induction of genetic mosaics in dividing cells.

In one example, the two guard cells of the stomata are daughters of a single precursor cell. To assay MC inheritance in this cell type, the epidermis of the leaf of a plant containing a MC is examined for the presence of the MC by the presence of a marker gene, including one encoding a fluorescent protein, a colored protein, a protein assayable by histochemical assay, or a gene affecting cell morphology. The number of loss events in which one guard cell contains the MC (L) and the number of cell divisions in which both guard cells contain the MC (B) are counted. The loss rate per cell division is determined as L/(L+B). Other lineage-based cell types are assayed in similar fashion.

Assay #4: Inheritance Assays on Modified Cells in the Presence of Chromosome Loss Agents

Assays #1-3 can be done in the presence of chromosome loss agents (e.g., colchicine, colcemid, caffeine, etopocide, nocodazole, oryzalin, and trifluran). It is likely that autonomous MCs are more susceptible to loss induced by chromosome loss agents; therefore, autonomous MCs show a lower rate of inheritance in the presence of chromosome loss agents. These methods have been used to study chromosome loss in fruit flies and yeast.

### G. Transformation of Cells

Various methods can be used to deliver DNA into cells. These include biological methods, (depending on the host) such as Agrobacterium, E. coli, and viruses; physical methods, such as biolistic particle bombardment, nanocopiea device, the Stein beam gun, silicon carbide whiskers and microinjection; electrical methods, such as electroporation; and chemical methods, such as the use of polyethylene glycol and other compounds that stimulate DNA uptake into cells (Dunwell 1999) and U.S. Pat. No. 5,464,765. These methods are well within the reach of one of skill in the art. Those of skill in the art can use, devise, and modify available procedures.

MC Transformation with Selectable Marker Gene

MC-modified cells in bombarded cells can often be isolated using a selectable marker gene. The bombarded tissues are transferred to a medium containing an appropriate selective agent. Tissues are transferred into selection. Selection of MC-modified cells can be further monitored by tracking fluorescent marker genes or by the appearance of modified explants (modified cells on explants can be green under light in selection medium, while surrounding non-modified cells are weakly pigmented).

Determination of MC Structure and Autonomy in Cells

The structure and autonomy of the MC in cells can be determined by: conventional and pulsed-field Southern blot hybridization to genomic DNA from modified tissue subjected or not subjected to restriction endonuclease digestion, dot blot hybridization of genomic DNA from modified tissue hybridized with different MC specific sequences, MC rescue, exonuclease activity, PCR on DNA from modified tissues with probes specific to the MC, or FISH to nuclei of modified cells. Table 2 below summarizes these methods.

TABLE 2
Autonomous MC assays

| Assay | Details | Potential outcome | Interpretation |
|---|---|---|---|
| Southern blot | Restriction digest | 1. Native sizes and pattern | 1. Autonomous or |
| | of genomic DNA | of bands | integrated via CEN |
| | compared to | | fragment |
| | purified MC | 2. Altered sizes or pattern | 2. Integrated or rearranged |
| | | of bands | |
| CHEF gel | Restriction digest | 1. Native sizes and pattern | 1. Autonomous or |
| Southern blot | of genomic DNA | of bands | integrated via CEN |
| | | | fragment |
| | | 2. Altered sizes or pattern | 2. Integrated or rearranged |
| | | of bands | |
| | Native genomic | 1. MC band migrating | 1. Autonomous circles or |
| | DNA (no digest) | ahead of genomic DNA | linears present |
| | | 2. MC band co-migrating | 2. Integrated |
| | | with genomic DNA | |
| | | 3. >1 MC bands observed | 3. Various possibilities |
| Exonuclease | Exonuclease | 1. Signal strength close to | 1. Autonomous circles |
| | digestion of | that w/o exonuclease | present |
| | genomic DNA with | 2. No signal or signal | 2. Integrated |
| | detection of | strength lower than w/o | |
| | circular MC by PCR, | exonuclease | |
| | dot blot, or | | |
| | restriction digest | | |
| | (optional), | | |
| | electrophoresis | | |
| | and southern blot | | |
| | (useful for circular | | |
| | MCs) | | |
| MC rescue | Transformation of | 1. Colonies isolated only | 1. Autonomous circles |
| | genomic DNA into | from MC cells with MC, | present, native MC |
| | E. coli followed by | not from controls; MC | structure. |
| | selection for | structure matches that of | |
| | antibiotic | the parental MC | |
| | resistance genes on | 2. Colonies isolated only | 2. Atuonomous circles |
| | MC | for MC cells with MCs, not | present, rearranged MC |
| | | from controls; MC | structure OR MCs |
| | | structure from parental | integrated via centromere |
| | | MC | fragment. |
| | | 3. Colonies in MC | 3. Various possibilities |
| | | modified plants and in | |
| | | controls | |
| PCR | PCR amplification | 1. All MC parts detected | 1. Complete MC sequences |
| | of various parts of | | present |
| | MC | 2. Subset of MC parts | 2. Partial MC sequences |
| | | detected | present |
| FISH | Detection of MC | 1. MC sequences | 1. Autonomous |
| | sequences in | detected, free of genome | |
| | mitotic or meiotic | 2. MC sequences | 2. Integrated |
| | nuclei by | detected, associated with | |
| | fluorescence in situ | genome | |
| | hybridization | 3. MC sequences | 3. Both autonomous and |
| | | detected, free and | integrated MC sequences |
| | | associated with genome | present |
| | | 4. No MC sequences | 4. MC DNA not visible by |
| | | detected | FISH |

Furthermore, MC structure can be examined by characterizing MCs rescued from MC-transformed cells. Circular MCs that contain bacterial sequences for their selection and propagation in bacteria can be rescued from a transformed cell and re-introduced into bacteria. If no loss of sequences has occurred during replication of the MC in cells, the MC is able to replicate in bacteria and confer antibiotic resistance. Total genomic DNA is isolated from the transformed cells. The purified genomic DNA is introduced into bacteria (e.g., E. coli), and the transformed bacteria are plated on solid medium containing antibiotics to select bacterial clones modified with MC DNA. Modified bacterial clones are grown, the plasmid DNA purified (by alkaline lysis for example), and DNA analyzed, such as by restriction enzyme digestion and gel electrophoresis or by sequencing.

### H. Analyses of Transformed Cells

MC Autonomy Demonstration by In Situ Hybridization

To assess whether the MC is autonomous from the native chromosomes, or has integrated into the native genome, in situ hybridizations can be used, such as FISH. In this assay, mitotic or meiotic tissue, possibly treated with metaphase arrest agents such as colchicines is obtained, and standard FISH methods are used to label both the centromere and sequences specific to the MC. Chromosomes are stained with a DNA-specific dye such as DAP1, Hoechst 33258, OliGreen, Giemsa YOYO, and TOTO. An autonomous MC is visualized as a body that shows hybridization signal with both centromere probes and MC specific probes and is separate from the native chromosomes.

Determination of Gene Expression Levels

The expression level of any gene present on the MC can be determined by several methods, such as for RNA, Northern Blot hybridization, Reverse Transcriptase-PCR, binding levels of a specific RNA-binding protein, in situ hybridization, or dot blot hybridization; or for proteins, Western blot hybridization, Enzyme-Linked Immunosorbant Assay (ELISA), fluorescent quantitation of a fluorescent gene product, enzymatic quantitation of an enzymatic gene product, immunohistochemical quantitation, or spectroscopic quantitation of a gene product that absorbs a specific wavelength of light.

Use of Exonuclease to Isolate Circular MC DNA from Genomic DNA

Exonucleases can be used to obtain pure MC DNA, suitable for isolation of MCs from E. coli or from cells. The method assumes a circular structure of the MC. A DNA preparation containing MC DNA and genomic DNA from the source organism is treated with exonuclease, for example lambda exonuclease combined with E. coli exonuclease I, or the ATP-dependent exonuclease (Qiagen, Inc.; Germantown, Md.). Because the exonuclease is only active on DNA ends, it specifically degrades the linear genomic DNA fragments, but does not degrade circular MC DNA. The result is MC DNA in pure form. The resultant MC DNA can be detected by a number of methods for DNA detection, such as PCR, dot blot, and Southern blot. Exonuclease treatment followed by detection of resultant circular MC can be used to determine MC autonomy.

Structural Analysis of MCs by Sequencing

Sequencing procedures, such as BAC-end sequencing (as appropriate), can be used to characterize MC clones for a variety of purposes, such as structural characterization, determination of sequence content, and determination of the precise sequence at a unique site on the chromosome (for example the specific sequence signature found at the junction between a centromere fragment and the vector sequences). In particular, this method is useful to prove the relationship between a parental MC and the MCs descended from it and isolated from plant cells by MC rescue, described above.

Methods for Scoring Meiotic MC Inheritance

A variety of methods can be used to assess the efficiency of meiotic MC transmission. In one embodiment of the method, gene expression of genes on the MC (marker genes or non-marker genes) can be scored by any method for detection of gene expression known to those skilled in the art, including visible scoring methods (e.g., fluorescence of fluorescent protein markers, scoring of visible phenotypes of the plant), scoring resistance of the cell or tissues to antibiotics, herbicides or other selective agents, measuring enzyme activity of proteins encoded by genes on the MC, measuring non-visible phenotypes, or directly measuring the RNA and protein products of gene expression using, for example, microarrays, northern blots, in situ hybridizations, dot blots, RT-PCR, western blots, immunoprecipitations, ELISAs, immunofluorescence and radio-immunoassays (RIAs). Gene expression or visible scoring of the MC markers can be scored in the post-meiotic stages.

FISH Analysis of MC Copy Number in Meiocytes and Cells

The copy number of the MC can be assessed in any cell or plant tissue by in situ hybridization, such as FISH. For example, FISH methods are used to label the centromere, using a probe that labels all chromosomes with one fluorescent tag, and to label sequences specific to the MC with another fluorescent tag. All centromere sequences are detected with the first tag; only MCs are detected with both the first and second tag. Nuclei are counter-stained with a DNA-specific dye, such as DAPI, Hoechst 33258, OliGreen, Giemsa YOYO, and TOTO. MC copy number is determined by counting the number of fluorescent foci that label with both tags.

### IV. Examples

The following examples are for illustrative purposes only and should not be interpreted as limitations of the claimed invention. There are a variety of alternative techniques and procedures available to those of skill in the art which would similarly permit one to successfully perform the intended invention.

The following examples illustrate the isolation and identification of centromere sequences in Zea mays. Zea mays centromere sequences are isolated and identified by immunoprecipitation of sheared, native chromatin with antisera raised against epitopes present Zea mays CenH3, called herein CenH3-3, CenH3a and CenH3b, and characterized by sequencing.

The following examples illustrate antibody production and chromatin preparation that can be used in the methods of the invention.

### Example 1

### Purified Antibodies Recognizing Zea mays CenH3

The following peptides were designed and synthesized in vitro for antiserum production:

| SEQ ID NO: | Sequence |
|---|---|
| 1 (CenH3-3) | GDSVKKTKPRH |
| 2 (CenH3a) | HQAVRKTAEKPKKKL |
| 3 (CenH3b) | LTNFVTNGKVERYTA |

These represent three different stretches of amino acids in the Z. mays CenH3 protein (e.g., Accession No. ACG39173).

These peptides were synthesized conjugated to keyhole limpet hemocyanin carrier protein. A cysteine was added to the C-terminus for coupling purposes and the peptide was acetylated at its N-terminus. The peptide was injected into rabbits at Affinity BioReagents (Golden, Colo.). Each rabbit was immunized over an 8 week period, bleeds tested by ELISA, and the rabbits finally exsanguinated, and the anti-CenH3 antibodies affinity purified. The yield for CenH3-3 was 29.9 mg; for CenH3a, 11.16 mg, and for CenH3b, 14.25 mg.

### Example 2

### ChIP in Zea mays (Prophetic)

Native ChIP is carried out from young leaves (^{˜}8-15 cm) or young roots (^{˜}1 wk after germination). Cells are incubated in TBS (0.01 M Tris-HCl [pH 7.5], 3 mM CaCl2, 2 mM MgCl2 with 0.1 mM phenylmethylsulphonyl fluoride [PMSF] and proteinase inhibitors) with 0.25% Tween40 at 4° C. on a roller stirrer for 2 h before extruding the nuclei using 30 strokes with the “Tight” or “A” prestle on a Dounce homogenizer (Wheaton). Nuclei are separated from cytoplasmic debris by centrifugation at 1500 g for 20 min at 4° C. through a 25%/50% discontinuous sucrose gradient. Oligonucleosomes are produced by digesting the nuclei with micrococcal nuclease (USB) in digestion buffer (0.32 M sucrose, 50 mM Tris-HCl at pH 7.5, 4 mM MgCl2, 1 mM CaCl2, 0.1 mM PMSF) at a concentration of 80 U/mg DNA at 37° C. for 10 min. The reaction mix is then centrifuged at 15,000 g at 4° C. The supernatant contains mainly mononucleosomes. The pellet fraction is further processed by incubation with lysis buffer (1 mM Tris-HCl at pH 7.5, 0.2 mM EDTA, 0.2 mM PMSF, and proteinase inhibitors) on ice for 1 h. The final supernatant containing oligonucleosomes is then obtained by centrifugation at 15,000 g for 5 min at 4° C. The two supernatant fractions are pooled and precleared by the incubation with 1:1000 dilution of the preimmunized rabbit serum and 1% protein A-sepharose (Amerham-Pharmcia) at 4° C. After preclearing, the supernatant is obtained by centrifugation at 250 g for 5 min at 4° C. This fraction is used immediately for immunoprecipitation (input fraction). Equal volumes of the supernatant and incubation buffer (50 mM NaCl, 20 mM Tris-HCl at pH 7.5, 5 mM EDTA, 0.1 mM PMSF, and protease inhibitors) are incubated with anti-CenH3 antibodies (either CenH3-3, CenH3a or CenH3b) a at 4° C. overnight. The immune complexes are then captured by incubating in 12.5% protein A-sepharose at 4° C. for 2 h. At the end of the incubation, the protein A-sepharose is washed extensively in a stepwise manner in buffer A (50 mM Tris-HCl at pH 7.5, 10 mM EDTA) containing 50, 100, and 150 mM NaCl. Bounded immune complexes are then eluted with 2 vol of 1% SDS.

DNA (bound fraction) is extracted from the eluate by phenol/chloroform/isoamyl alcohol extraction and prepared for high-throughput sequencing and analysis for centromere sequences as detailed in the present disclosure.

Alternatively, RNase-free DNase I is used for chromatin digestion. Alternatively, the chromatin is crosslinked before immunoprecipitation.

### CITED NON-PATENT LITERATURE

Alonso, A., R. Mahmood, et al. (2003). “Genomic microarray analysis reveals distinct locations for the CENP-A binding domains in three human chromosome 13q32 neocentromeres.” Hum Mol Genet. 12(20): 2711-2721.
Ausubel, F. M. (1987). Current protocols in molecular biology. Brooklyn, N.Y. Media, Pa., Greene Publishing Associates; J. Wiley, order fulfillment.
Baumann, C., R. Korner, et al. (2007). “PICH, a centromere-associated SNF2 family ATPase, is regulated by Plk1 and required for the spindle checkpoint.” Cell 128(1): 101-114.
Bhattacharya, D. and L. Medlin (1998). “Algal phylogeny and the origin of land plants.” Plant Physiol 116: 9-15.
Cai, M. and R. W. Davis (1990). “Yeast centromere binding protein CBF1, of the helix-loop-helix protein family, is required for chromosome stability and methionine prototrophy.” Cell 61(3): 437-446.
Carlson, S. R., G. W. Rudgers, et al. (2007). “Meiotic transmission of an in vitro-assembled autonomous maize minichromosome.” PLoS Genet. 3(10): 1965-1974.
Casas-Mollano, J. A., K. van Dijk, et al. (2007). “SET3p monomethylates histone H3 on lysine 9 and is required for the silencing of tandemly repeated transgenes in Chlamydomonas.” Nucleic Acids Res 35(3): 939-950.
Collas, P. (2010). “The current state of chromatin immunoprecipitation.” Mol Biotechnol 45(1): 87-100.
Connelly, C. and P. Hieter (1996). “Budding yeast SKP1 encodes an evolutionarily conserved kinetochore protein required for cell cycle progression.” Cell 86(2): 275-285.
Cooke, C. A., M. M. Heck, et al. (1987). “The inner centromere protein (INCENP) antigens: movement from inner centromere to midbody during mitosis.” J Cell Biol 105(5): 2053-2067.
Cottarel, G., J. H. Shero, et al. (1989). “A 125-base-pair CEN6 DNA fragment is sufficient for complete meiotic and mitotic centromere functions in Saccharomyces cerevisiae.” Mol Cell Biol 9(8): 3342-3349.
Dai, J., B. A. Sullivan, et al. (2006). “Regulation of mitotic chromosome cohesion by Haspin and Aurora B.” Dev Cell 11(5): 741-750.
De Martino, A., A. Amato, et al. (2009). “Mitosis in diatoms: rediscovering an old model for cell division.” BioEssays 31: 874-884.
Diaz-Martinez, L. A., J. F. Gimenez-Abian, et al. (2007). “Regulation of centromeric cohesion by sororin independently of the APC/C.” Cell Cycle 6(6): 714-724.
Doe, C. L., G. Wang, et al. (1998). “The fission yeast chromo domain encoding gene chp1(+) is required for chromosome segregation and shows a genetic interaction with alpha-tubulin.” Nucleic Acids Res 26(18): 4222-4229.
Dunleavy, E. M., A. L. Pidoux, et al. (2007). “A NASP (N1/N2)-related protein, Sim3, binds CENP-A and is required for its deposition at fission yeast centromeres.” Mol Cell 28(6): 1029-1044.
Dunwell, J. M. (1999). “Transformation of maize using silicon carbide whiskers.” Methods Mol Biol 111: 375-382.
Earnshaw, W. C. and B. R. Migeon (1985). “Three related centromere proteins are absent from the inactive centromere of a stable isodicentric chromosome.” Chromosoma 92(4): 290-296.
Foltz, D. R., L. E. Jansen, et al. (2009). “Centromere-specific assembly of CENP-a nucleosomes is mediated by HJURP.” Cell 137(3): 472-484.
Foltz, D. R., L. E. Jansen, et al. (2006). “The human CENP-A centromeric nucleosome-associated complex.” Nat Cell Biol 8(5): 458-469.
Freeman-Cook, L. L., J. M. Sherman, et al. (1999). “The Schizosaccharomyces pombe hst4(+) gene is a SIR2 homologue with silencing and centromeric functions.” Mol Biol Cell 10(10): 3171-3186.
Garner, M. M. and A. Revzin (1981). “A gel electrophoresis method for quantifying the binding of proteins to specific DNA regions: application to components of the Escherichia coli lactose operon regulatory system.” Nucleic Acids Res 9(13): 3047-3060.
Greaves, I. K., D. Rangasamy, et al. (2007). “H2A.Z contributes to the unique 3D structure of the centromere.” Proc Natl Acad Sci USA 104(2): 525-530.
Hagstrom, K. A., V. F. Holmes, et al. (2002). “C. elegans condensin promotes mitotic chromosome architecture, centromere organization, and sister chromatid segregation during mitosis and meiosis.” Genes Dev 16(6): 729-742.
He, D., C. Zeng, et al. (1998). “CENP-G: a new centromeric protein that is associated with the alpha-1 satellite DNA subfamily.” Chromosoma 107(3): 189-197.
Hori, T., M. Amano, et al. (2008). “CCAN makes multiple contacts with centromeric DNA to provide distinct pathways to the outer kinetochore.” Cell 135(6): 1039-1052.
Jiang, W., K. Middleton, et al. (1993). “An essential yeast protein, CBF5p, binds in vitro to centromeres and microtubules.” Mol Cell Biol 13(8): 4884-4893.
Jin, W., J. C. Lamb, et al. (2005). “Molecular and functional dissection of the maize B chromosome centromere.” Plant Cell 17(5): 1412-1423.
Jin, W., J. R. Melo, et al. (2004). “Maize centromeres: organization and functional adaptation in the genetic background of oat.” Plant Cell 16(3): 571-581.
King, M. C., T. G. Drivas, et al. (2008). “A network of nuclear envelope membrane proteins linking centromeres to microtubules.” Cell 134(3): 427-438.
Kitajima, T. S., S. A. Kawashima, et al. (2004). “The conserved kinetochore protein shugoshin protects centromeric cohesion during meiosis.” Nature 427(6974): 510-517.
Klein, F., P. Mahr, et al. (1999). “A central role for cohesins in sister chromatid cohesion, formation of axial elements, and recombination during yeast meiosis.” Cell 98(1): 91-103.
Lechner, J. and J. Carbon (1991). “A 240 kd multisubunit protein complex, CBF3, is a major component of the budding yeast centromere.” Cell 64(4): 717-725.
Lee, H. R., W. Zhang, et al. (2005). “Chromatin immunoprecipitation cloning reveals rapid evolutionary patterns of centromeric DNA in Oryza species.” Proc Natl Acad Sci USA 102(33): 11793-11798.
Lo, A. W., D. J. Magliano, et al. (2001). “A novel chromatin immunoprecipitation and array (CIA) analysis identifies a 460-kb CENP-A-binding neocentromere DNA.” Genome Res 11(3): 448-457.
Lorence, A. and R. Verpoorte (2004). “Gene transfer and expression in plants.” Methods Mol Biol 267: 329-350.
Maddox, P. S., F. Hyndman, et al. (2007). “Functional genomics identifies a Myb domain-containing protein family required for assembly of CENP-A chromatin.” J Cell Biol 176(6): 757-763.
Maruyama, S., H. Kuroiwa, et al. (2007). “Centromere dynamics in the primitive red alga Cyanidioschyzon merolae.” Plant J 49(6): 1122-1129.
Maruyama, S., M. Matsuzaki, et al. (2008). “Centromere structures highlighted by the 100%-complete Cyanidioschyzon merolae genome.” Plant Signal Behav 3(2): 140-141.
Meluh, P. B. and D. Koshland (1995). “Evidence that the MIF2 gene of Saccharomyces cerevisiae encodes a centromere protein with homology to the mammalian centromere protein CENP-C.” Mol Biol Cell 6(7): 793-807.
Nagaki, K. and M. Murata (2005). “Characterization of CENH3 and centromere-associated DNA sequences in sugarcane.” Chromosome Res 13(2): 195-203.
Nagaki, K., J. Song, et al. (2003). “Molecular and cytological analyses of large tracks of centromeric DNA reveal the structure and evolutionary dynamics of maize centromeres.”Genetics 163(2): 759-770.
Nagaki, K., P. B. Talbert, et al. (2003). “Chromatin immunoprecipitation reveals that the 180-bp satellite repeat is the key functional DNA element of Arabidopsis thaliana centromeres.” Genetics 163(3): 1221-1225.
Nishihashi, A., T. Haraguchi, et al. (2002). “CENP-I is essential for centromere function in vertebrate cells.” Dev Cell 2(4): 463-476.
Noutoshi, Y., R. Arai, et al. (1997). “Designing of plant artificial chromosome (PAC) by using the Chlorella smallest chromosome as a model system.” Nucleic Acids Symp Ser (37): 143-144.
Ogiwara, H., T. Enomoto, et al. (2007). “The INO80 chromatin remodeling complex functions in sister chromatid cohesion.” Cell Cycle 6(9): 1090-1095.
Okada, M., K. Okawa, et al. (2009). “CENP-H-containing complex facilitates centromere deposition of CENP-A in cooperation with FACT and CHD1.” Mol Biol Cell 20(18): 3986-3995.
Okano, M., D. W. Bell, et al. (1999). “DNA methyltransferases Dnmt3a and Dnmt3b are essential for de novo methylation and mammalian development.” Cell 99(3): 247-257.
Papait, R., C. Pistore, et al. (2007). “Np95 is implicated in pericentromeric heterochromatin replication and in major satellite silencing.” Mol Biol Cell 18(3): 1098-1106.
Phelps-Durr, T. L. and J. A. Birchler (2004). “An asymptotic determination of minimum centromere size for the maize B chromosome.” Cytogenet Genome Res 106(2-4): 309-313.
Rattner, J. B., A. Rao, et al. (1993). “CENP-F is a .ca 400 kDa kinetochore protein that exhibits a cell-cycle dependent localization.” Cell Motil Cytoskeleton 26(3): 214-226.
Saitoh, S., K. Takahashi, et al. (1997). “Mis6, a fission yeast inner centromere protein, acts during G1/S and forms specialized chromatin required for equal segregation.” Cell 90(1): 131-143.
Saunders, W. S., C. Chue, et al. (1993). “Molecular cloning of a human homologue of Drosophila heterochromatin protein HP1 using anti-centromere autoantibodies with anti-chromo specificity.” J Cell Sci 104 (Pt 2): 573-582.
Schittenhelm, R. B., F. Althoff, et al. (2010). “Detrimental incorporation of excess Cenp-A/Cid and Cenp-C into Drosophila centromeres is prevented by limiting amounts of the bridging factor Cal1.” J Cell Sci 123(Pt 21): 3768-3779.
Siu, F. K., L. T. Lee, et al. (2008). “Southwestern blotting in investigating transcriptional regulation.” Nat Protoc 3(1): 51-58.
Stoler, S., K. Rogers, et al. (2007). “Scm3, an essential Saccharomyces cerevisiae centromere protein required for G2/M progression and Cse4 localization.” Proc Natl Acad Sci USA 104(25): 10571-10576.
Sugata, N., E. Munekata, et al. (1999). “Characterization of a novel kinetochore protein, CENP-H.”J Biol Chem 274(39): 27343-27346.
Tadeu, A. M., S. Ribeiro, et al. (2008). “CENP-V is required for centromere organization, chromosome alignment and cytokinesis.” Embo J 27(19): 2510-2522.
Uren, A. G., L. Wong, et al. (2000). “Survivin and the inner centromere protein INCENP show similar cell-cycle localization and gene knockout phenotype.” Curr Biol 10(21): 1319-1328.
Urh, M., D. Hartzell, et al. (2008). “Methods for detection of protein-protein and protein-DNA interactions using HaloTag.” Methods Mol Biol 421: 191-209.
Vafa, O. and K. F. Sullivan (1997). “Chromatin containing CENP-A and alpha-satellite DNA is a major component of the inner kinetochore plate.” Curr Biol 7(11): 897-900.
van Steensel, B. and S. Henikoff (2000). “Identification of in vivo DNA targets of chromatin proteins using tethered dam methyltransferase.” Nat Biotechnol 18(4): 424-428.
Verdel, A., S. Jia, et al. (2004). “RNAi-mediated targeting of heterochromatin by the RITS complex.” Science 303(5658): 672-676.
Vernarecci, S., P. Ornaghi, et al. (2008). “Gcn5p plays an important role in centromere kinetochore function in budding yeast.” Mol Cell Biol 28(3): 988-996.
Wang, H., W. Tang, et al. (2002). “A chromatin immunoprecipitation (ChIP) approach to isolate genes regulated by AGL15, a MADS domain protein that preferentially accumulates in embryos.” Plant J 32(5): 831-843.
Williams, B. C., M. Gatti, et al. (1996). “Bipolar spindle attachments affect redistributions of ZW10, a Drosophila centromere/kinetochore component required for accurate chromosome segregation.” J Cell Biol 134(5): 1127-1140.
Yen, T. J., D. A. Compton, et al. (1991). “CENP-E, a novel human centromere-associated protein required for progression from metaphase to anaphase.” Embo J 10(5): 1245-1254.
Zhong, C. X., J. B. Marshall, et al. (2002). “Centromeric retroelements and satellites interact with maize kinetochore protein CEN H3.” Plant Cell 14(11): 2825-2836.

1. A method of identifying a centromere sequence, comprising:
(a) immunoprecipitating protein-DNA complexes from fragmented chromatin derived from at least one cell using an antibody to a centromere-associated protein;
(b) separately sequencing individual nucleic acid molecules of a population of nucleic acid molecules isolated from the protein-DNA complexes;
(d) calculating the frequency of occurrence of each nucleic acid sequence in the population; and
(e) identifying a nucleic acid molecule sequence which has an increased frequency of occurrence in the population as a centromere sequence.

2. A method of identifying a centromere sequence, comprising:
(a) fusing a centromere-associated protein with a DNA adenine methyltransferase to create a fusion protein;
(b) expressing the fusion protein in at least one cell of interest;
(c) isolating methylated DNA from the cell of interest;
(d) separately sequencing the isolated methylated DNA; and
(e) identifying the DNA which has an increased frequency of occurrence as a centromere sequence.

3. A method of identifying a centromere sequence, comprising:
(a) fusing a centromere-associated protein with a protein that tightly binds to a chloroalkane resin to create a fusion protein;
(b) expressing the fusion protein in at least one cell of interest;
(c) isolating chromatin from the cell of interest and cross-linking the isolated chromatin;
(d) isolating fusion protein/DNA complexes by passing the isolated, cross-linked chromatin over a chrloroalkane resin and reversing the cross-linking of the resin to disrupt the protein/DNA complexes; and
(e) separately sequencing the isolated DNA; and
(f) identifying the DNA which has an increased frequency of occurrence as a centromere sequence.

4. A method of identifying a centromere sequence, comprising:
(a) labeling and isolating DNA from at least one cell of interest;
(b) incubating the labeled and isolated DNA with a centromere-associated protein, forming centromere-associated protein/DNA complexes;
(c) electrophoresing the mixture from step (b) to separate the centromere-associated protein/DNA complexes from unbound labeled DNA;
(d) isolating slower-migrating DNA representing centromere-associated protein/DNA complexes;
(e) isolating the DNA from the centromere-associated protein/DNA complexes;
(f) separately sequencing the isolated DNA; and
(g) identifying the DNA which has an increased frequency of occurrence as a centromere sequence.

5. A method of identifying a centromere sequence, comprising:
(a) immobilizing a centromere-associated protein onto a substrate;
(b) incubating labeled DNA isolated from at least one cell of interest with the centromere-associated protein;
(c) isolating bound DNA;
(d) separately sequencing the isolated DNA; and
(e) identifying the DNA which has an increased frequency of occurrence as a centromere sequence.

6. The method of any of claims 1-5, further comprising, prior to sequencing the nucleic acid or DNA, separately amplifying individual nucleic acid molecules of a population of nucleic acid molecules isolated from the protein-DNA complexes.

7. The method of any of claims 1-5, wherein at least one cell is at least one plant, fungal, algal, or protist cell.

8. The method of claim 7, wherein at least one cell is at least one algal cell.

9. The method of claim 8 wherein at least one algal cell is of the Chlorophyceae, Pluerastrophyceae, Ulvophyceae, Micromonadophyceae, or Charophytes class.

10. The method of claim 9, wherein at least one algal cell is a cell of an alga of the Chlorophyceae class.

11. The method of claim 10, wherein at least one algal cell is a cell of an alga of the Dunaliellale, Volvocale, Chloroccale, Oedogoniale, Sphaerolpleale, Chaetophorale, Microsporale, or Tetrasporale orders.

12. The method of claim 11, wherein at least one algal cell is a cell of an Amphora, Ankistrodesmus, Asteromonas, Botryococcus, Chaetoceros, Chlamydomonas, Chlorococcum, Chlorella, Cricosphaera, Crypthecodinium, Cyclotella, Dunaliella, Emiliania, Euglena, Haematococcus, Halocafeteria, Isochrysis, Monoraphidium, Nannochloris, Nannochloropsis, Navicula, Neochloris, Nitzschia, Ochromonas, Oedogonium, Oocystis, Ostreococcus, Pavlova, Phaeodactylum, Pleurochrysis, Pleurococcus, Pyramimonas, Scenedesmus, Skeletonema, Stichococcus, Tetraselmis, Thalassiosira or Volvox species.

13. The method of claim 7, wherein at least one cell is at least one fungal cell.

14. The method of claim 13, wherein at least one fungal cell is a cell of a chytrid, blastocladiomycete, neocallimastigomycete, zgomycete, trichomycete, glomeromycote, ascomycete, or basidiomycete.

15. The method of claim 13 wherein at least one fungal cell is a cell of a glomerocyote, ascomycete, or basidiomycete.

16. The method of any of claims 1-5, wherein the centromere-associated protein is selected from the group consisting of centromere proteins, centromere protein-recruitment proteins, and kinetochore proteins.

17. The method of any of claims 1-5, wherein the centromere-associated protein is selected from the group consisting of Cal1, Cbf1, Cbf3, Cbf5, CenH3 (Cenp-A), Cenp-B, Cenp-C, Cenp-D, Cenp-E, Cenp-F, Cenp-G, Cenp-H, Cenp-I, Cenp-K, Cenp-L, Cenp-M, Cenp-N, Cenp-O, Cenp-P, Cenp-Q, Cenp-R, Cenp-S, Cenp-T, Cenp-U, Cenp-V, Cenp-W, Chd1, Chp1, cohesin, condensin, Dnmt3b, Fact, Gcn5p, H2A.Z, Haspin, Hjurp, HP1, Hst4, Ima1, Incep, Ino80, Kms2, Knl-2, Mif2, Mis6, Np95, Pich, Sad1, Scm3, Shugoshin, Sim3, Skp1, Sororin, Survivin, Tas3, ZW10, and homologs thereof.

18. The method of claim 17, wherein the centromere-associated protein is CenH3 or a homolog of CenH3.

19. The method of claim 1, further comprising performing one or more assays to evaluate the centromere sequence.

20. The method of claim 19, wherein at least one assay is an assay for stable heritability of an artificial chromosome comprising the centromere sequence.

21. The method of claim 19, wherein at least one assay detects the presence of a selectable or nonselectable marker on an artificial chromosome comprising the centromere sequence.

22. The method of claim 19, wherein at least one assay detects the presence of the centromere sequence or a nucleic acid sequence linked thereto on an artificial chromosome.

23. A recombinant nucleic acid molecule comprising a centromere sequence identified by the method of any of claims 1-5, wherein the centromere sequence is not adjacent to one or more sequences positioned adjacent to the centromere sequence in the genome from which the centromere sequence is derived.

24. An artificial chromosome comprising a centromere sequence identified by the method of any of claims 1-5.

25. The artificial chromosome of claim 24, further comprising at least one selectable or nonselectable marker.

26. The artificial chromosome of claim 24, further comprising at least one gene encoding a structural protein, a regulatory protein, an enzyme, a ribozyme, an antisense RNA, an shRNA, or an siRNA.

27. A cell comprising an artificial chromosome of claim 24.

28. A method of identifying an algal centromere sequence, comprising:
(a) immunoprecipitating protein-DNA complexes from fragmented chromatin derived from at least one algal cell using an antibody to a centromere-associated protein; and
(b) sequencing nucleic acid molecules isolated from the protein-DNA complexes to identify an algal centromere sequence.

29. The method of claim 28, wherein the method does not require addition of a cross-linking agent prior to immunoprecipitating protein-DNA complexes from the fragmented chromatin.

30. The method of claim 29, wherein the method does not require hybridizing a nucleic acid molecule isolated from the immunoprecipitated protein-DNA complexes to one or more known centromere sequences.

31. The method of claim 28, wherein at least one algal cell is at least one green, yellow-green, brown, golden brown, or red algal cell.

32. The method of claim 31, wherein at least one algal cell is an algal cell of the Chlorophyceae class.

33. The method of claim 31, wherein at least one algal cell is an algal cell of the Dunaliellale, Volvocale, Chloroccale, Oedogoniale, Sphaerolpleale, Chaetophorale, Microsporale, or Tetrasporale order.

34. The method of claim 33, wherein at least one algal cell is a cell of an Amphora, Ankistrodesmus, Aster vmonas, Botryococcus, Chaetoceros, Chlamydomonas, Chlorococcum, Chlorella, Cricosphaera, Crypthecodinium, Cyclotella, Dunaliella, Emiliania, Euglena, Haematococcus, Halocafeteria, Isochrysis, Monoraphidium, Nannochloris, Nannochloropsis, Navicula, Neochloris, Nitzschia, Ochromonas, Oedogonium, Oocystis, Ostreococcus, Pavlova, Phaeodactylum, Pleurochrysis, Pleurococcus, Pyramimonas, Scenedesmus, Skeletonema, Stichococcus, Tetraselmis, Thalassiosira or Volvox species.

35. The method of claim 28, wherein the centromere-associated protein is selected from the group consisting of centromere proteins, centromere protein-recruitment proteins, and kinetochore proteins.

36. The method of claim 28 wherein the centromere-associated protein is selected from the group consisting of Cal1, Cbf1, Cbf3, Cbf5, CenH3 (Cenp-A), Cenp-B, Cenp-C, Cenp-D, Cenp-E, Cenp-F, Cenp-G, Cenp-H, Cenp-I, Cenp-K, Cenp-L, Cenp-M, Cenp-N, Cenp-O, Cenp-P, Cenp-Q, Cenp-R, Cenp-S, Cenp-T, Cenp-U, Cenp-V, Cenp-W, Chd1, Chp1, cohesin, condensin, Dnmt3b, Fact, Gcn5p, H2A.Z, Haspin, Hjurp, HP1, Hst4, Ima1, Incep, Ino80, Kms2, Knl-2, Mif2, Mis6, Np95, Pich, Sad1, Scm3, Shugoshin, Sim3, Skp1, Sororin, Survivin, Tas3, ZW10, and homologs thereof.

37. The method of claim 36, wherein the centromere-associated protein is CenH3 or a homolog of CenH3.

38. The method of claim 37, wherein the antibody specifically binds to the N terminus of CenH3 or the N terminus of a homolog of CenH3.

39. The method of claim 28, further comprising amplifying the nucleic acid molecules isolated from the immunoprecipitated protein-DNA complexes prior to sequencing.
